(19) United States
(12) Patent Application Publication
(10) Pub. No.: US 2025/0140338 A1
(43) Pub. Date: May 1, 2025

(54) DNA METHYLATION-BASED CANCER DIAGNOSTICS

(71) Applicant: The USA, as represented by the Secretary, Department of Health and Human Services, Bethesda, MD (US)

(72) Inventors: Kenneth Aldape, Bethesda, MD (US); Ziedulla Abdullaev, Bethesda, MD (US); Rustamzhon Turakulov, Bethesda, MD (US); Antonios Papanicolau-Sengos, Bethesda, MD (US); Stefania Pittaluga, Bethesda, MD (US); Mark Raffeld, Bethesda, MD (US); Elaine Sarkin Jaffe, Bethesda, MD (US); Omkar Singh, Bethesda, MD (US)

(21) Appl. No.: 18/499,833

(22) Filed: Nov. 1, 2023

(51) Int. Cl. G16B 20/00; C12Q 1/6886; G16B 40/30; G16H 20/00; G16H 50/20

(52) U.S. Cl. CPC G16B 20/00; C12Q 1/6886; G16B 40/30; G16H 20/00; G16H 50/20; C12Q 2600/112; C12Q 2600/154

(57) ABSTRACT

Disclosed herein are methods for classifying a tumor using a trained classifier. The method may include providing a methylation profile to a classifier trained to identify tumor classes using unsupervised clustering, generating a classification of the tumor based on the methylation profile and a reference set, where the reference set is generated from training the classifier, generating a confidence score based on the correlation of the methylation profile to the classification from the classifier, and updating the classifier and reference set with the methylation profile and classification. The classifier may include a plurality of family sub-classifiers and/or a plurality of class sub-classifiers.

### GOVERNMENT INTEREST STATEMENT

The present subject matter was made with U.S. government support. The U.S. government has certain rights in this subject matter.

### FIELD

The present disclosure relates to systems and methods for classifying tumors based on DNA methylation. More specifically, the disclosure relates to DNA methylation-based cancer diagnostics for tumors of the central nervous system, kidney and hematopoietic system.

### BACKGROUND

Appropriate management of patients with cancer depends critically on accurate pathological diagnosis. Substantial effort is put into the standardization of cancer diagnosis to promote uniformity of processes, but this has proven challenging, as substantial inter-observer variability remains in the histologic diagnosis of many tumor types.

The classification of renal neoplasms is currently based on histology, immunohistochemical stains (IHC) and, for a small subset of familial cases, genetic findings. A great deal of heterogeneity exists in the pathology, biology and clinical behavior of neoplasms affecting the kidney. The 2022 WHO classification of renal tumors contains over 40 benign and malignant renal neoplasms including an “unclassified” category with overlapping or ambiguous morphologic features (WHO). Challenging subsets of renal neoplasms include microscopically similar neoplasms such as hybrid tumors, chromophobe renal cell carcinomas (ChRCCs), and benign oncocytomas, all of which have eosinophilic cytoplasm but distinct prognoses, and subsets of ccRCC and pRCCs that have a particularly poor prognosis. Advances in the understanding of the molecular biology of major renal cell carcinoma (RCC) cancer types have been accomplished by efforts such as the Cancer Genome Atlas (TCGA) and others. While specific types of renal neoplasms exhibit distinct histopathology and, in many cases, can be readily distinguished and defined as a specific tumor type, often with the help of immunohistochemical markers, it is known that genomic markers can also contribute the classification in specific instances. For example, genomic differences between the 3 most common major classes of kidney cancers (clear cell RCC, papillary RCC, and chromophobe RCC) have been described. However, some cases and some tumor types may be more challenging for accurate diagnosis and could be subject to inter-observer variability on histopathology.

The developmental complexity and heterogeneity of hematolymphoid (HL) neoplasms reflect the vast array of distinct tumor entities defined in the current classifications of HL neoplasms. The tumors are clinically and biologically diverse encompassing a wide spectrum from tumors that can be followed by conservative management or surgery (e.g., primary cutaneous follicle center lymphoma) to highly malignant tumors responding poorly to any therapy (e.g., extranodal NK/T cell lymphoma). There is high interobserver variability in the histopathological diagnosis of many HL tumors as reported by previous studies. To address this, molecular features have been introduced in the recent classifications of HL neoplasms, but mainly for selected entities including myeloid neoplasms and acute leukemias. The current diagnosis for many entities, especially for lymphoid neoplasms, is still largely based on morphology and immunophenotyping, along with ancillary genomic studies when required and available. In addition, for some entities defined by specific gene fusions, it may not be possible to identify the fusion adequately by FISH or RNA-based methods for a variety of technical and specimen-related limitations. The diagnostic discordance and subjectivity may confound decision-making in clinical practice and interfere with the interpretation and validity of clinical trial results. A precise diagnosis of histopathological subtypes of HL neoplasms thus has important implications for the choice of therapy and patient outcomes and novel approaches are needed to bridge these gaps.

Central nervous system (CNS) tumors represent a diverse spectrum of neoplasms that originate within the brain or spinal cord. Especially challenging CNS neoplasms include morphologically similar entities such as glioblastomas, which are the most common and aggressive type of brain tumor; diffuse midline glioma, H3 K27-altered, an aggressive pediatric type of brain tumor, ependymoma, which can be either benign or malignant, the latter can be difficult to treat and have a poor prognosis. Accurate classification of these tumors is essential for determining prognosis and guiding treatment strategies. Traditional classification methods, such as histopathology, immunohistochemistry, and molecular methods have limitations in distinguishing between different CNS tumor subtypes. Interpretation of histological images and testing results is subject to inter-observer discordance among pathologists. These factors may have significant implications for patient management. Therefore, there is a growing need for more precise and objective diagnostic tools.

DNA methylation involves the addition of a methyl group to the cytosine residue of CpG dinucleotides in the genome, leading to epigenetic modifications that can alter gene expression patterns. Aberrant DNA methylation patterns are a hallmark of many cancer types, including kidney, CNS, and HL tumors, making them attractive candidates for molecular classification. DNA methylation signatures in neoplastic diseases represent a combination of signatures that is derived from the normal tissue cell of origin of that neoplasm, in combination with specific epigenetic changes that occur in the process of tumorigenesis. Genome wide methylation profiling has been shown as a robust and reproducible means of identifying and classifying tumors of a variety of organ systems. In particular, methylation classifiers for tumors of the central nervous system as well as soft tissue sarcomas have been described. These classifiers and the methylation platform have specific advantages in the clinic whereby they are amenable to tumor tissues in the format commonly used by most pathology laboratories, i.e., formalin fixed paraffin embedded tissues. The widespread use of a common platform (methylation arrays) facilitates relatively seamless coalescence of cohorts across centers for large-scale studies. Beyond the mere interest of methylation-based classification schemes, there is value in the development of data-driven platforms as a diagnostic adjunct in cancer. The current standard of care for most cancer diagnoses in the clinic is inspection of histopathology from a biopsies or resected tissue specimen, performed by pathologists. Despite extensive training in diagnostic pathology, this process remains subjective to some extent and, especially in difficult cases is subject to inter-observer discordance among pathologists, difficulties of which have been reported to extend to renal neoplasms. One clinically important problem is in the interpretation of renal biopsies, where the result determines the surgical approach to resection. In particular, a biopsy result of oncocytoma would trigger a more conservative surgical approach compared to the diagnosis of a higher-grade lesion, yet diagnostic difficulties for an oncocytoma designation are known to exist.

Therefore, there is a need for a method of using methylation signatures to automatically classify distinct families, classes, and/or sub-classes to distinguish and classify a larger variety of tumors to provide a more accurate diagnosis and improved treatment protocols.

### SUMMARY

This disclosure provides systems and methods of classifying a tumor using a trained classifier.

Provided herein is a system for classifying a tumor, the system including: a processor in communication with a memory, the memory including instructions executable by the processor to: receive a methylation profile of the tumor; provide the methylation profile to a classifier trained to identify tumor classes using unsupervised clustering; generate a classification of the tumor based on the methylation profile and a reference set, wherein the reference set is generated from training the classifier; generate a confidence score based on the correlation of the methylation profile to the classification from the classifier; and update the classifier and reference set with the methylation profile and classification.

In some aspects, the classifier includes a plurality of sub-classifiers. For example, the classifier can include a plurality of family sub-classifiers for separate functional regions of the methylation profile. The classifier can include at least 5 family sub-classifiers. The memory can further include instructions executable by the processor to: generate a family consistency score and a family mean calibrated score from the sub-classifiers. The memory can further include instructions executable by the processor to: generate a family classification based on the family consistency score and the family mean calibrated score. The classifier can further include a class sub-classifier for each family. The classifier can include at least 5 class sub-classifiers. The memory can further include instructions executable by the processor to: generate a class consistency score and a class mean calibrated score from the class sub-classifiers. The memory can further include instructions executable by the processor to: generate a class and/or sub-class classification based on the class consistency score and the class mean calibrated score. The confidence score can include a mean calibrated score of the family consistency score, the family mean calibrated score, the class consistency score, and/or the class mean calibrated score.

In some aspects, the memory further includes instructions executable by the processor to: identify a tumor family, class, and sub-class based on the classification of the tumor. The tumor sub-class can be identified based on clusters of characteristics identified by the classifier. The tumor can be a renal tumor, hematolymphoid tumor, or CNS tumor. When the confidence score is above a threshold, there is high confidence in the classification. The threshold can be at least 0.9.

In some aspects, when the confidence score is below 0.5 or the classifier cannot generate a classification, the memory further includes instructions executable by the processor to: generate an alert for a new class or sub-class. The memory can further include instructions executable by the processor to: evaluate the methylation profile, a sample of the tumor, orthogonal DNA and/or RNA data, and/or patient demographics to generate the new class or sub-class. The memory can further include instructions executable by the processor to: update the reference set with the new class or sub-class. The memory can further include instructions executable by the processor to: re-train the classifier with the updated reference set.

In some aspects, the unsupervised clustering uses uniform manifold approximation and projection (UMAP) dimensionality reduction and/or additional dimensionality reduction methodologies. The memory can further include instructions executable by the processor to: train the classifier prior to providing the methylation profile.

Further disclosed herein is a method that can include providing a methylation profile to a classifier trained to identify tumor classes using unsupervised clustering, generating a classification of the tumor based on the methylation profile and a reference set, generating a confidence score based on the correlation of the methylation profile to the classification from the classifier, and updating the classifier and reference set with the methylation profile and classification. The reference set can be generated from training the classifier.

In various aspects, the classifier includes a plurality of sub-classifiers. For example, the classifier can include a plurality of family sub-classifiers for separate functional regions of the methylation profile. The classifier can include at least 5 family sub-classifiers. In some aspects, the method can further include generating a family consistency score and a family mean calibrated score from the sub-classifiers. The family classification can then be generated based on the family consistency score and the family mean calibrated score.

In some aspects, the classifier can further include a class sub-classifier for each family. For example, the classifier can include at least 5 class sub-classifiers. In some aspects, the method can further include generating a class consistency score and a class mean calibrated score from the class sub-classifiers. A class and/or sub-class classification can then be generated based on the class consistency score and the class mean calibrated score. In an aspect, the confidence score can include a mean calibrated score of the family consistency score, the family mean calibrated score, the class consistency score, and/or the class mean calibrated score.

In some aspects, the method can further include identifying a tumor family, class, and sub-class based on the classification of the tumor. The tumor sub-class can be identified based on clusters of characteristics identified by the classifier. The tumor can be a renal tumor, hematolymphoid tumor, or CNS tumor.

In some aspects, when the confidence score is above a threshold, there is high confidence in the classification. The threshold can be at least 0.9. When the confidence score is below 0.5 or the classifier cannot generate a classification, the method can include generating an alert for a new class or sub-class. In an aspect, the method can further include evaluating the methylation profile, a sample of the tumor, orthogonal DNA and/or RNA data, and/or patient demographics to generate the new class or sub-class. The method can then further include updating the reference set with the new class or sub-class.

In another aspect, the unsupervised clustering uses uniform manifold approximation and projection (UMAP) dimensionality reduction and/or additional dimensionality reduction methodologies. In some aspects, the method can further include training the classifier prior to providing the methylation profile.

Further provided herein is a method of recommending a treatment plan for the patient. The method can include diagnosing the tumor using the generated classification. In some aspects, the method can further include forming a treatment plan specific to the diagnosis of the tumor. In additional aspects, the method can further include comparing the classification to a histological and/or molecular evaluation of the tumor. The classification can be adjusted based on the comparison. For example, the classification can be adjusted based on demographic data or other DNA and/or RNA data of the patient. In some aspects, the method can further include treating the patient based on the treatment plan or classification. The treatment can include treatment steps specific for the class or sub-class of tumor.

Further provided herein is a method of training a classifier for classifying a tumor. The method can include providing a reference methylation dataset comprising a plurality of methylation profiles for a plurality of samples, applying unsupervised clustering to the methylation dataset, filtering the samples based on clustering of the methylation profiles to identify families of the tumor, applying unsupervised clustering using the most variable probes from different functional regions on each methylation profile in the methylation dataset for a plurality of sub-classifiers to identify classes and sub-classes of the tumor based on clusters of methylation profiles, and generating a reference set based on the identified families, classes, and sub-classes.

In some aspects, the unsupervised clustering uses Uniform Manifold Approximation and Projection (UMAP) dimensionality reduction and/or additional dimensionality reduction methodologies. The different functional regions are selected from the group consisting of Gene body, Island, Opensea, Other genomic region, Open chromatin region, Shore, Shelf, TSS, 5′UTR, and whole array probes.

In an aspect, the method can further include implementing a support vector machine (SVM) algorithm for each classifier. In another aspect, the method can further include applying a 5 by 5 nested cross-validation (CV). In additional aspects, the method can further include calibrating each classifier. In further aspects, the method can further include updating the classification database with new methylation profiles of new samples.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The description will be more fully understood with reference to the following figures and data graphs, which are presented as various embodiments of the disclosure and should not be construed as a complete recitation of the scope of the disclosure. It is noted that, for purposes of illustrative clarity, certain elements in various drawings may not be drawn to scale. Understanding that these drawings depict only exemplary embodiments of the disclosure and are not therefore to be considered to be limiting of its scope, the principles herein are described and explained with additional specificity and detail through the use of the accompanying drawings in which:

FIG. 1 is an example classification method in one embodiment.

FIG. 2 illustrates example system embodiments.

FIG. 3 illustrates an example machine learning environment.

FIG. 4 illustrates an example training and validation workflow. Concordance is based on family scores (not class scores).

FIG. 5A shows a UMAP of a reference set. FIG. 5B shows an oncoplot of critical genetic events. FIG. 5C shows a survival plot of clear cell entities. FIG. 5D shows a survival plot of papillary entities. FIG. 5E shows an overall survival plot of eosinophilic entities.

FIG. 6A shows Cibersort CD14 immune proportions in the ccRCC classes.

FIG. 6B shows overall survival of cases with the top and bottom third proportion of CD14. FIG. 6C shows progression free survival of cases with the top and bottom third proportion of CD14. FIG. 6D shows Methylcibersort immune cell proportions per class.

FIG. 7 shows a histology review of 146 cases from the reference set. For cases where the pathologist and methylation label did not agree, available orthogonal molecular information were provided.

FIG. 8 shows a confusion matrix of internal validation dataset (n=302) with high specific class score (at least 0.84).

FIG. 9 shows a Sankey plot of cases with high class score from Discovery-Unclassified dataset. The right nodes are the predicted methylation classes. The connecting ribbon color indicates whether the provided and methylation labels are concordant.

FIG. 10 shows a Sankey plot of cases with high score from Discovery-Distant dataset. The original reported diagnosis is in the left node. The right nodes are the predicted methylation classes. The connecting ribbon color indicates whether the initial diagnosis and the methylation class labels are concordant after evaluation of orthogonal data.

FIG. 11 shows a Sankey plot of cases with high score from Evelonn dataset. The reported diagnosis is in the left node (all diagnoses in the Evelonn dataset are ccRCC). The right nodes are the predicted specific-class methylation level diagnoses. The connecting ribbon color indicates whether the provided and methylation labels are concordant.

FIG. 12 shows a Sankey plot of cases with high score from the Chopra dataset. The reported diagnoses are in the left node. The right nodes are the predicted specific-class methylation level diagnoses. The connecting ribbon color indicates whether the provided and methylation labels are concordant.

FIG. 13 illustrates an example of a stratified classification methodology implemented.

FIG. 14 shows a comparison of cases which received >0.84 confidence score in at least one classifier.

FIG. 15A and FIG. 15B show two example cases where the Bethesda classifier output is a high score and the DKFZ classifier output is a low score.

FIG. 16 shows non-negative matrix factorization as an example approach to discover new tumor entities.

FIG. 17 shows an example of development of a methylation-based classifier for hematolymphoid neoplasms.

FIG. 18A shows an overview of the 41 Hematolymphoid tumor methylation classes and 3 non-neoplastic tissue methylation classes of the reference cohort.

FIG. 18B shows unsupervised clustering of reference cohort samples (n=1,156) using UMAP dimensionality reduction. Individual samples are color-coded in the respective class color (n=44) and labelled with the class abbreviation. The color code and abbreviations are identical to FIG. 18A.

FIG. 18C shows methylation-based classes with distinct clinical outcomes. MCL_SOX11N_B shows significantly better survival than MCL_SOX11P and MCL_SOX11N_A. TLL_A shows significantly poor survival than TLL_B. AML_KMT2A shows significantly poor survival than AML_RUNX1, AML_CEBPA and AML_CBFB.

FIG. 19A is an overview of the 7 methylation class families based on similar methylation profiles.

FIG. 19B is an example schematic of the classifier showing it is composed of a family level classifier and a class level classifier with a mean calibrated score for both.

FIG. 20A illustrates testing samples on external validation set.

FIG. 20B illustrates testing samples on an exploration set.

FIG. 20C shows a combined independent test set of 483 independent hematolymphoid samples.

FIG. 20D shows a heat map showing results of classifier incorporating n=482 biologically independent samples allocated to 44 methylation classes (one sample “discrepant-not validated”, with discrepant results that could not be reviewed to re-evaluate diagnosis was excluded from this analysis). Deviations from the bisecting line represent misclassification errors.

FIG. 21 shows a comparison of pathological diagnosis and methylation class prediction.

FIG. 22 is a doughnut chart showing the 8 cases with methylation results discrepant from institutional original diagnosis which on re-evaluation lead to change in diagnosis with the new diagnosis in accordance with the methylation classifier prediction. The inner doughnut chart shows the original diagnosis, and the outer doughnut chart shows the new changed diagnosis (in agreement with methylation class).

FIG. 23A is a box and whisker plot showing the effect of tumor purity on classifier performance. The samples that are a classifier match (cross threshold of family mean score>0.94 and class mean score>0.86) have a significantly higher median tumor purity of 0.67 using RF_purify_estimate in comparison to cases that are no classifier match and don't cross the threshold with a median tumor purity of 0.61. (p value<0.00005).

FIG. 23B shows the dataset(n=482) sorted according to tumor purity using RF_purify_estimate.

FIG. 24 shows an example flow chart of a clinical implementation of example 4.

FIG. 25 provides a table of the methylation classes identified using the classifier of example 4.

FIG. 26A shows the unsupervised clustering of the reference cohort samples using UMAP dimensionality reduction of classes of B-cell lymphomas.

FIG. 26B shows survival analysis of the MCL subgroups.

FIG. 26C shows copy number analysis from the methylation data for the MCL subgroups.

FIG. 27A shows the unsupervised clustering of the reference cohort samples without PTCL, NOS using UMAP dimensionality reduction of classes of T-cell lymphomas.

FIG. 27B shows unsupervised clustering of samples of peripheral T-cell lymphoma, NOS (PTCL, NOS) using UMAP dimensionality reduction.

FIG. 28A shows unsupervised clustering of the reference cohort samples (except B and T-cell lymphomas) using UMAP dimensionality reduction of classes of other HL neoplasms.

FIG. 28B shows survival analysis of acute myeloid leukemia subgroups and T lymphoblastic leukemia/lymphoma subgroups.

FIG. 29 shows the unsupervised clustering of hematolymphoid reference cohort samples using UMAP dimensionality reduction.

FIG. 30A shows morphology and immunohistochemistry (IHC) images that were acquired using standard techniques and a table of markers that the biopsy sample was positive or negative for.

FIG. 30B shows results of the molecular analysis of the biopsy, including small nucleotide variants.

FIGS. 30C and 30D show the results of the methylation classifier, which classified the biopsy as a malignant lymphoma c/w extranodal NK/T-cell lymphoma.

FIG. 31 shows the results of the methylation classifier, which classified the biopsy as a myeloid sarcoma/chloroma.

Reference characters indicate corresponding elements among the views of the drawings. The headings used in the figures do not limit the scope of the claims.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and such references mean at least one of the embodiments.

Reference to “one embodiment”, “an embodiment”, or “an aspect” means that a particular feature, structure, or characteristic described in connection with the embodiment is included in at least one embodiment of the disclosure. The appearances of the phrase “in one embodiment” or “in one aspect” in various places in the specification are not necessarily all referring to the same embodiment, nor are separate or alternative embodiments mutually exclusive of other embodiments. Moreover, various features are described which may be exhibited by some embodiments and not by others.

As used herein, “about” refers to numeric values, including whole numbers, fractions, percentages, etc., whether or not explicitly indicated. The term “about” generally refers to a range of numerical values, for instance, ±0.5-1%, ±1-5% or ±5-10% of the recited value, that one would consider equivalent to the recited value, for example, having the same function or result.

The terms “classifier”, “trained classifier”, and “sub-classifier” may be used interchangeably to refer to all or part of software executed by a processor that is trained using machine learning techniques to generate a tumor classification based on a methylation profile of a tumor sample.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any example term. Likewise, the disclosure is not limited to various embodiments given in this specification.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims or can be learned by the practice of the principles set forth herein.

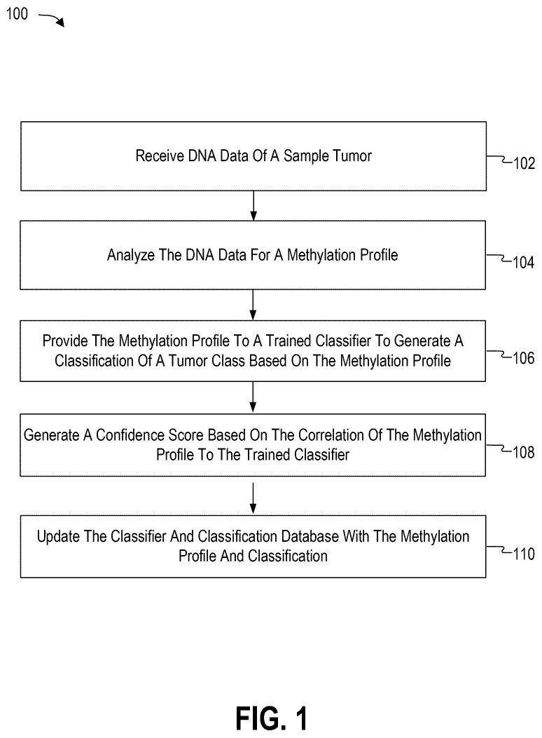

Provided herein are methods of classifying a tumor and methods of treatment thereof using a classifier based on a DNA methylation profile. The classifier can be a trained classifier such that the classification method is performed automatically upon input of DNA data or a methylation profile. The classifier can include multiple sub-classifiers to improve the speed and accuracy of the classification. The classifier provides for classification of a tumor to a sub-class level without relying on subjectivity from a pathologist or other physician. A more accurate identification of the tumor sub-class can then lead to better clinical outcomes, e.g., classification specific treatment. In addition, the classifier can identify previously un-identified and clinically relevant tumor types and subtypes. The overall framework of the classifier method 100 is shown in FIG. 1.

At step 102, the classifier method 100 can include receiving DNA data of a sample of the tumor. The tumor can be any tumor associated with cancer. Non-limiting examples of tumors include but are not limited to renal tumors, CNS tumors, hematolymphoid (HL) neoplasms, tumors of the lung, GI tract tumors, reproductive system tumors, or any other tumors. For example, the classifier can be used in pan-cancer analysis. In some examples, a biopsy (sample) of the tumor can be acquired from a patient. The sample can be formalin fixed paraffin embedded tissues. The DNA data can be acquired from the sample using any techniques known in the art. Step 102 may be optional if the DNA data has already been acquired. In this aspect, the method 100 may start at steps 104 or 106.

At step 104, the classifier method 100 can include analyzing the DNA data for a methylation profile. Step 104 may be optional if the methylation profile has already been acquired. In some examples, the method 100 can start with receiving a methylation profile of a DNA sample of the tumor that has previously been acquired/analyzed. The methylation profile can be obtained from the DNA data using any techniques known in the art. For example, the methylation profile can be obtained using a 450 k methylation array, an EPIC/850 k methylation array. In some examples, the methylation profile may be in the form of raw intensity data files (idat).

At step 106, the classifier method can include providing the methylation profile to a trained classifier for classifying the tumor to generate a classification of a tumor class based on the methylation profile and a reference set. The reference set can be stored in a classification database. In some examples, the method may include generating a family classification, a class classification, a sub-class classification, or combinations thereof. The reference set is generated from training the classifier. The reference set can include methylation profiles and classifications of samples used to train the classifier or are later input into the classifier. In some examples, as further described herein, the classification database/reference set is generated, and the classifier is trained using unsupervised clustering of methylation profiles of reference samples using UMAP dimensionality reduction. The classifier is trained prior to providing the methylation profile. The classification database/reference set can be updated with new methylation profiles that are input into the classifier with their classification. The updated reference set may be used to re-train the classifier.

The classification can be performed automatically once the methylation profile is provided to the classifier. The classifier may be previously trained using known methylation profiles of tumors. In some aspects, the trained classifier can be trained with known methylation profile datasets using unsupervised clustering. The unsupervised clustering may use hierarchical clustering, uniform manifold approximation, projection for dimension reduction (UMAP), and/or additional dimensionality reduction methodologies. The data can then be filtered based on the clustering within coherent methylation families and classes. In some examples, the data is stored in the classification database as the reference set.

The classifier may include one or more sub-classifiers. The classifier may include a plurality of family sub-classifiers and/or a plurality of class sub-classifiers. The one or more sub-classifiers can be specific for separate functional regions of the methylation profile, a particular family, or class. In an example, the classifier can include a plurality of family sub-classifiers and a plurality of class sub-classifiers.

In some examples, the classifier can include a plurality of family sub-classifiers to generate a family classification. Each family sub-classifier can be for separate functional regions of the methylation profile. The different functional regions can be selected from the group consisting of Gene body, Island, Opensea, Other genomic region, Open chromatin region, Shore, Shelf, TSS, 5′UTR, and whole array probes. In some examples, 10,000 to 200,000 of the most variable probes can be used for the sub-classifier for each region.

The classifier can include at least 1, at least 2, at least 3, at least 4, at least 5, at least 6, at least 7, at least 8, at least 9, or at least 10 family sub-classifiers.

In additional examples, the classifier can include a class sub-classifier for each family that is identified from the family sub-classifiers. The class sub-classifiers can generate a class and/or sub-class classification. The classifier can include at least 1, at least 2, at least 3, at least 4, at least 5, at least 6, at least 7, at least 8, at least 9, at least 10, at least 15, at least 20, at least 30, at least 40, at least 50 class sub-classifiers.

In some examples, the trained classifier may be trained to identify at least 20, at least 25, at least 30, at least 35, at least 40, at least 45, or at least 50 clusters based on the known methylation profiles. In an example, each cluster can correlate to a family and/or class. The identification of a larger number of classes/sub-classes can allow for a more specific classification that then can lead to a more specific and productive treatment. Because treatment plans can vary based on the class of tumor, it is important to have as specific a classification as possible. In various embodiments, the classifier may be trained to identify at least 20, at least 25, at least 30, at least 35, at least 40, at least 45, at least 50, at least 60, at least 70, at least 80, at least 90, at least 100, at least 120, at least 140, at least 160, at least 180, or at least 200 classes or sub-classes. For example, when the tumor is a renal tumor, the classifier may have been trained to identify at least 20 kidney neoplastic and non-neoplastic DNA methylation classes. In another example, when the tumor is a CNS tumor, the classifier may have been trained to identify at least 50 classes. In a further example, when the tumor is a hematolymphoid tumor the classifier may have been trained to identify at least 30 classes. Each class may be considered a “bin” based on clustering of the training data. In some examples, the method may further include generating a bin-based consistency score for each class.

The classes may include sub-classes or be divided into sub-classes. In some aspects, the method can further include identifying a tumor sub-class/subtype based on the classification of the tumor. The tumor sub-class can be identified based on clusters of characteristics identified by the trained classifier. In an example, when the tumor is a renal tumor, tumor classes or sub-classes may include but are not limited to ccRCC (A, B, C, D, E, or like), pccRCC, pRCC (A, B, C, or D), FH-deficient, Reverse polarity, ChRCC (A, B, or cluster 1), hybrid, oncocytoma, TSC/MTOR oncocytic, CCSK, MRT, Wilms, metanephric adenoma, renal angiomyolipoma, cortex, medulla, and urothelial clusters (REFs).

In other examples when the tumor is a CNS tumor, tumor classes or sub-classes may include but are not limited to Adamantinous craniopharyngioma, Adult-type diffuse high grade glioma, IDH-wildtype (subtype B, subtype E, or subtype F), Anaplastic neuroepithelial tumor with condensed nuclei, Angiocentric glioma, MYB/MYBL1-altered, Astroblastoma, MN1-altered, MN1:BEND2-fused, Astrocytoma, IDH-mutant (high grade or lower grade), Atypical malignant peripheral nerve sheath tumor, Atypical teratoid/rhabdoid tumor (MYC-subtype, SHH-subtype, or TYR-subtype), Cauda equina neuroendocrine tumor [paraganglioma], subtype non-CIMP, Central neurocytoma, Cerebellar liponeurocytoma, Chordoid glioma, PRKCA-mutant, Chordoma, Choroid plexus carcinoma (adult subtype or pediatric subtype), Choroid plexus papilloma (adult subtype or pediatric subtype), CIC-rearranged sarcoma, CNS embryonal tumor with BRD4:LEUTX fusion, CNS Embryonal tumor with PLAG-family amplification, CNS neuroblastoma, FOXR2-activated, CNS Schwannoma, VGLL-fused, CNS tumor with BCOR internal tandem duplication, Control tissue (cerebellum, cerebral hemisphere, corpus callosum, hypothalamus, optic pathway, pineal gland, pituitary gland (anterior lobe), pons, reactive tumor microenvironment, or white blood cells), Cribriform neuroepithelial tumor, SMARCB1-altered, Desmoplastic infantile ganglioglioma/desmoplastic infantile astrocytoma, Desmoplastic myxoid tumor, SMARCB1-altered, Diffuse astrocytoma, MYB or MYBL1-altered (subtype B [infratentorial], subtype C [isomorphic], or subtype D), Diffuse glioneuronal tumor with oligodendroglioma-like features and nuclear clusters (DGONC), Diffuse hemispheric glioma, H3 G34-mutant, Diffuse IDH-wildtype glioma with FGFR3-TACC3 fusion, Diffuse large B-cell lymphoma of the CNS, Diffuse leptomeningeal glioneuronal tumor (subtype 1 or subtype 2), Diffuse midline glioma, H3 K27-altered (subtype EGFR-altered, or subtype H3 K27-mutant or EZHIP expressing), Diffuse pediatric-type high grade glioma (H3 wildtype and IDH wildtype Subtype A or Subtype B, MYCN subtype, RTK1 subtype, subclass A, B, or C, or RTK2 subtype, subclass A or B), Dysembryoplastic neuroepithelial tumor, Embryonal tumor with multilayered rosettes (C19MC-altered or non-C19MC-altered), Ependymoma, posterior fossa group A (PFA) (subclass 1a, 1b, 1c, 1d, 1e, 1f, 2a, 2b, or 2c), Ependymoma, posterior fossa group B (PFB) (subclass 1, 2, 3, 4, or 5), Ependymoma, spinal, Ependymoma, spinal, MYCN-amplified, Ependymoma, supratentorial, YAP1 fusion-positive, Ependymoma, supratentorial, ZFTA fusion-positive, subtype ZFTA-RELA fused (subclass A, B, C, D, or E), Ewing sarcoma, Extraventricular neurocytoma, Ganglioglioma/Polymorphous low-grade neuroepithelial tumor of the young, Germinoma (subtype KIT mutant or subtype KIT wildtype), Glial neoplasm with BCOR/BCORL1 fusion, Glioblastoma, IDH-wildtype (primitive neuronal component, mesenchymal subtype, mesenchymal subtype, atypical, RTK1 subtype, RTK2 subtype, subtype posterior fossa, Glioneuronal tumor, subtype A, Hemangioblastoma, High grade glioma with pleomorphic and pseudopapillary features (HPAP), High-grade astrocytoma with piloid features, Infant-type hemispheric glioma, Inflammatory microenvironment, Intracranial mesenchymal tumor, Intraocular medulloepithelioma, Langerhans cell histiocytosis, Malignant melanotic nerve sheath tumor, Malignant peripheral nerve sheath tumor, Medulloblastoma, non-WNT/non-SHH, (Group 3 subtype (subclass I, II, III, or IV) or Group 4 subtype (subclass V, VI, VII, or VIII), Medulloblastoma, SHH-activated (IDH-mutant, subclass 1, 2, 3, or 4), Medulloblastoma, WNT-activated, Medullomyoblastoma, Melanoma metastasis, Meningioma (clear cell subtype (SMARCE1-altered), subtype benign (subclass 1, 2, or 3), subtype intermediate (subclass A or B), or subtype malignant), Myxoid glioneuronal tumor, PDGFRA-mutant, Myxopapillary ependymoma, Neuroblastoma (ALT/TERT TMM positive, MYCN type, or TMM negative), Neuroepithelial tumor (PATZ1 fusion, MN1:CXXC5-fused, or PLAGL1-fused), Olfactory neuroblastoma, IDH-wildtype, Oligodendroglioma, IDH-mutant and 1p/19q-codeleted, Oligosarcoma, IDH-mutant, Papillary craniopharyngioma, Papillary glioneuronal tumor; PRKCA-fused, Papillary tumor of the pineal region (subtype A or B), Pilocytic astrocytoma (infratentorial, infratentorial, subclass FGFR1-altered, posterior fossa, subtype A, supratentorial midline, or ganglioglioma, hemispheric), Pineal parenchymal tumor of intermediate differentiation, KBTBD4-altered (subtype A or B), Pineoblastoma, MYC/FOXR2-activated, Pineoblastoma (subtype miRNA processing altered 1 (subclass A or B) or altered 2 or subtype RB1-altered (pineal retinoblastoma)), Pineocytoma, Pituicytoma, granular cell tumor of the sellar region, and spindle-cell oncocytoma, Pituitary adenoma (subtype ACTH-producing, subtype gonadotrophin-producing, subtype prolactin-producing, subtype STH-producing (subclass densely granulated A, subclass densely granulated B, or subclass sparsely granulated), or subtype TSH-producing), Plasmacytoma of the CNS, Pleomorphic xanthoastrocytoma, Plexiform neurofibroma, Primary CNS circumscribed melanocytic tumor, Primary intracranial sarcoma, DICER1-mutant, Retinoblastoma, Retinoblastoma, subtype MYCN-altered, Rhabdomyosarcoma (alveolar subtype, embryonal subtype, or MYOD1-mutant), Rosette forming glioneuronal tumor, Schwannoma, Sinonasal undifferentiated carcinoma, IDH2-mutant, Solitary fibrous tumor, Subependymal giant cell astrocytoma, TSC1/TSC2-altered, Subependymoma and ependymoma, posterior fossa, Subependymoma, spinal (subtype A or B), Subependymoma, supratentorial, Teratoma, or Yolk sac tumor.

In other examples, when the tumor is an HL tumor, tumor classes or sub-classes may include but are not limited to myeloid, B-cell lymphoid, T-cell lymphoid, Hs/dn, primary myelofibrosis, juvenile myelomonocytic leukemia, myelodysplastic syndrome (high risk), acute myeloid leukemia (RUNX1-RUNX1T1, NUP98 fusions, KMT2A fusions, CEBPA mutation, CBFB-MYH11, or IDH mutation), B-lymphoblastic leukemia/lymphoma (subgroup A or subgroup B), chronic lymphocytic leukemia, mantle cell lymphoma (subgroup A, subgroup B, or subgroup C), hairy cell leukemia, plasma cell neoplasms, Marginal zone lymphoma (subgroup A or subgroup B), follicular lymphoma (subgroup A or subgroup B), diffuse large B-cell lymphoma (subgroup A or subgroup B), T-lymphoblastic leukemia/lymphoma (subgroup A or subgroup B), follicular helper T-cell lymphoma, anaplastic large cell lymphoma, hepatosplenic T-cell lymphoma, extranodal T-cell lymphoma (group A, group B, or group C), extranodal NK/T leukemia/lymphoma, Sezary syndrome, adult T-cell leukemia/lymphoma, histiocytic sarcoma, Langerhans cell histiocytosis, or follicular dendritic cell sarcoma.

In some aspects, classifying the tumor can further include identifying patient demographic data or orthogonal DNA and/or RNA data and adjusting the classification of the tumor based on the demographic data or orthogonal DNA data. In some examples, the demographic data or the orthogonal DNA and/or RNA data may confirm the classification based only on the methylation profile. In other examples, the orthogonal DNA and/or RNA data may change the classification based on the methylation profile. The demographic data or orthogonal DNA and/or RNA data may include DNA copy number patterns, sex, age, or other related DNA or RNA data. The orthogonal DNA and/or RNA data can be previously identified as information associated with certain classifications.

At step 108, the classifier method may include generating a confidence score based on the correlation of the methylation profile to the classification from the classifier. Again, because treatment plans may vary based on classification, a physician would find it beneficial to have as accurate a classification as possible. The confidence score may provide information as to the confidence of the classification. For example, a confidence score of 0.9 or greater may indicates a high confidence in the classification of the tumor. In various examples, a confidence score of greater than or equal to 0.84, 0.85, 0.86, 0.87, 0.88, 0.89, 0.9, 0.91, 0.92, 0.93, 0.94, or 0.95 indicates a high confidence in the classification of the tumor. In some examples, a confidence score of 0.5 to 0.84 can indicate a suggestive classification. With a suggestive classification, the consideration of orthogonal DNA and/or RNA data may contribute to increasing the confidence of the classification.

The method can include generating a confidence score for a family classification, a class classification, a sub-class classification, or combinations thereof. Multiple confidence scores may be combined together to produce an overall confidence score.

In some examples, the method can further include generating a family consistency score and/or a family mean calibrated score from the sub-classifiers. A family classification can be generated based on the family consistency score and/or the family mean calibrated score. In additional examples, the method can further include generating a class consistency score and/or a class mean calibrated score from the class sub-classifiers. A class and/or sub-class classification can be generated based on the class consistency score and/or the class mean calibrated score. The confidence score can include a combination of a mean calibrated score of the family consistency score, the family mean calibrated score, the class consistency score, and/or the class mean calibrated score. In some examples, the confidence score can be a numerical value. For example, the confidence score is the mean of the family consistency score, the family mean calibrated score, the class consistency score, and/or the class mean calibrated score. In other examples, the confidence score can be an interpretation of a numerical value. Table 1 shows interpretations of a combination of the family mean calibrated score, the family consistency score, the class mean calibrated score, and the class consistency score 5 example conditions.

TABLE 1

| | | family_{——} | | class_{——} | |
|---|---|---|---|---|---|
| | family_score | consistency_{—} | class_{—} | consistency_{—} | Interpretation |
| Condition | mean | score | Score_mean | score | (confidence score) |
| Condition 1 | >0.9 | 1.0 | >0.9 | 0.9-1.0 | Superfamily - match/ |
| | | | | | Class - match |
| Condition 2 | >0.9 | 1.0 | 0.5-0.9 | any | Superfamily - match/ |
| | | | | | Class - suggestive |
| Condition 3 | 0.5-0.9 | any | 0.5-1 | any | Superfamily - suggestive/ |
| | | | | | Class - suggestive |
| Condition 4 | 0.5-0.9 | any | <0.5 | any | Superfamily - suggestive/ |
| | | | | | Class no match |
| Condition 5 | <0.5 | any | any | any | Superfamily - no match/ |
| | | | | | Class no match |

When the confidence score is below 0.5 or the classifier cannot generate a classification, the method can include generating an alert for a new class or sub-class. For example, if the presented methylation profile does not fall within any pre-identified families, classes, or sub-classes in the classification database, then an alert can be generated. This may prompt further investigation to identify a new family, class, or sub-class for a rare type. Additional patient demographic data, orthogonal DNA data, orthogonal RNA data, or other histologic data can be used to develop the new family, class, or sub-class. Then, the new family, class, or sub-class can be used to update the classification database.

At step 110, the method may include updating the classifier and classification database with the methylation profile and classification. The classifier can be updated and improved by adding new patient data with each use and/or refining class designations based on new information. In some aspects, updating the classifier can include re-training the classifier using the updated classification database/reference set.

Further provided herein is a method of recommending a treatment plan for the patient by diagnosing the tumor using the classification generated from the trained classifier. The method can include forming a treatment plan specific to the diagnosis of the tumor. In some aspects, the classification can be compared to histological and/or molecular evaluation of the tumor sample prior to diagnosing the tumor. The classification can also be compared to demographic data or other orthogonal DNA and/or RNA data of the patient. The histological and/or molecular evaluation, demographic data, and/or other orthogonal DNA and/or RNA data can be used to confirm the classification or adjust the classification of the tumor. Adjusting the classification can depend on the confidence score generated by the method.

The method can further include treating the patient based on the recommended treatment plan or classification. The treatment can be performed over a period of time sufficient to reduce or remove the tumor. The treatment can include treatment steps specific for the class or sub-class of tumor. For example, the treatment can include administering a chemotherapy drug, surgery, radiation, hormone therapy, immunotherapy, bone marrow transplantation, and/or any cancer therapy known in the art. The treatment can include combining various treatments over a period of time sufficient to reduce or remove the tumor.

Further provided herein is a method of training a classifier for classifying a tumor. As further described herein, the method can include providing a reference methylation dataset comprising a plurality of methylation profiles for a plurality of samples, applying unsupervised clustering to the methylation dataset, filtering the samples based on clustering of the methylation profiles to identify families of the tumor, applying unsupervised clustering using the most variable probes from different functional regions on each methylation profile in the methylation dataset for a plurality of sub-classifiers to identify classes and sub-classes of the tumor based on clusters of methylation profiles, and generating a classification database based on the identified families, classes, and sub-classes. The unsupervised clustering can use Uniform Manifold Approximation and Projection (UMAP) dimensionality reduction and/or additional dimensionality reduction methodologies.

The different functional regions can be selected from the group consisting of Gene body, Island, Opensea, Other genomic region, Open chromatin region, Shore, Shelf, TSS, 5′UTR, and whole array probes. In some examples, 10,000 to 200,000 of the most variable probes can be used for the sub-classifier for each region.

As further described below, in some examples, the method can further include implementing a support vector machine (SVM) algorithm for each classifier. In additional examples, the method can further include applying a 5 by 5 nested cross-validation (CV). In further examples, the method can further include calibrating each classifier. In even further examples, the method can further include updating the classification database with new methylation profiles of new samples.

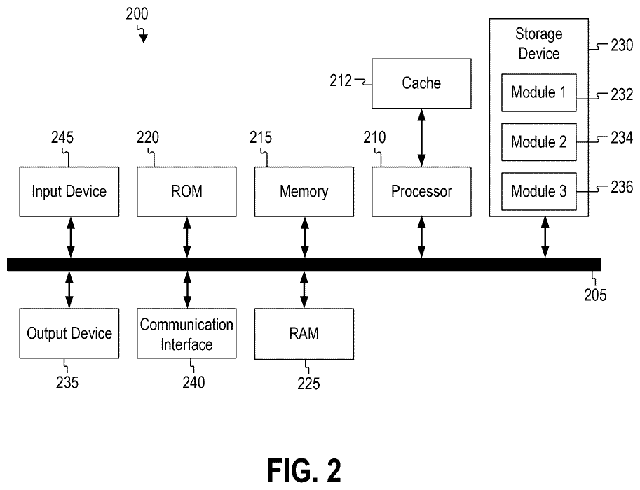

The disclosure now turns to the example system illustrated in FIG. 2 which may be used to implement the methods for classifying a tumor, diagnosing a tumor, and/or training the classifier. FIG. 2 shows an example of computing system 200 in which the components of the system are in communication with each other using connection 205. Connection 205 can be a physical connection via a bus, or a direct connection into processor 210, such as in a chipset or system-on-chip architecture. Connection 205 can also be a virtual connection, networked connection, or logical connection.

In some examples computing system 200 is a distributed system in which the functions described in this disclosure can be distributed within a datacenter, multiple datacenters, a peer network, throughout layers of a fog network, etc. In some examples, one or more of the described system components represents many such components each performing some or all of the function for which the component is described. In some examples, the components can be physical or virtual devices.

Example system 200 includes at least one processing unit (CPU or processor) 210 and connection 105 that couples various system components including system memory 215, read only memory (ROM) 220 or random access memory (RAM) 225 to processor 210. Computing system 200 can include a cache of high-speed memory 212 connected directly with, in close proximity to, or integrated as part of processor 210.

Processor 210 can include any general purpose processor and a hardware service or software service, such as services 232, 234, and 236 stored in storage device 230, configured to control processor 210 as well as a special-purpose processor where software instructions are incorporated into the actual processor design. Processor 210 may essentially be a completely self-contained computing system, containing multiple cores or processors, a bus, memory controller, cache, etc. A multi-core processor may be symmetric or asymmetric.

To enable user interaction, computing system 200 includes an input device 245, which can represent any number of input mechanisms, such as a microphone for speech, a touch-sensitive screen for gesture or graphical input, keyboard, mouse, motion input, speech, etc. Computing system 200 can also include output device 235, which can be one or more of a number of output mechanisms known to those of skill in the art. In some instances, multimodal systems can enable a user to provide multiple types of input/output to communicate with computing system 200. Computing system 200 can include communications interface 240, which can generally govern and manage the user input and system output, and also connect computing system 200 to other nodes in a network. There is no restriction on operating on any particular hardware arrangement and therefore the basic features here may easily be substituted for improved hardware or firmware arrangements as they are developed.

Storage device 230 can be a non-volatile memory device and can be a hard disk or other types of computer readable media which can store data that are accessible by a computer, such as magnetic cassettes, flash memory cards, solid state memory devices, digital versatile disks, cartridges, battery backed random access memories (RAMs), read only memory (ROM), and/or some combination of these devices.

The storage device 230 can include software services, servers, services, etc., that when the code that defines such software is executed by the processor 210, it causes the system to perform a function. In some examples, a hardware service that performs a particular function can include the software component stored in a computer-readable medium in connection with the necessary hardware components, such as processor 210, connection 205, output device 235, etc., to carry out the function.

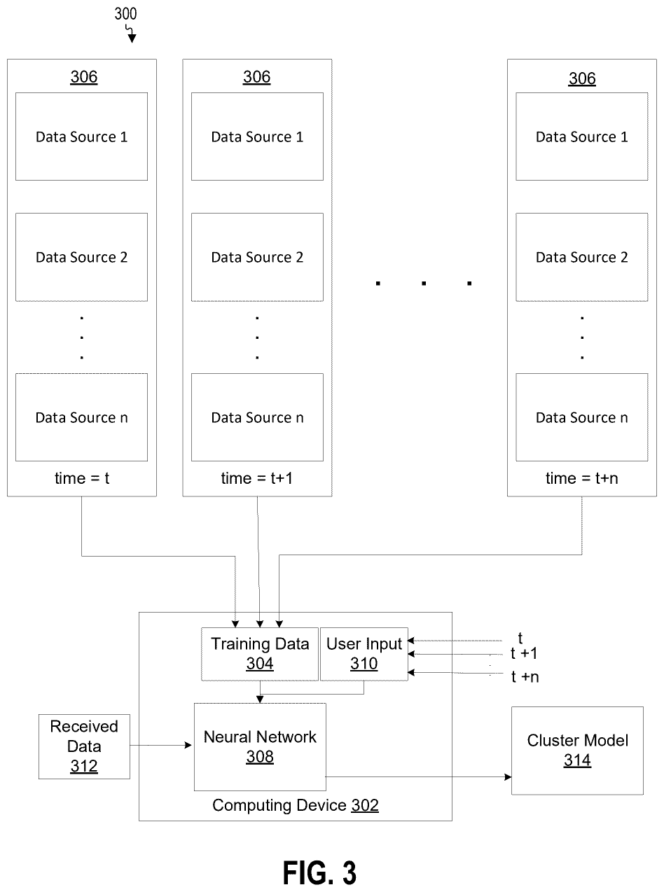

The disclosure now turns to FIG. 3, which illustrates an example machine learning environment 300. The machine learning environment can be implemented on one or more computing devices 302 (e.g., cloud computing servers, virtual services, distributed computing, one or more servers, etc.). The computing device(s) 302 can include training data 304 (e.g., one or more databases or data storage device, including cloud-based storage, storage networks, local storage, etc.). In some examples, the training data may be used as an input into an unsupervised learning model to identify clusters (families, classes, sub-classes) of similar data. In other examples, the training data may include data from labeled DNA methylation data, DNA copy number patterns, sex, age, histological labels, and/or molecular profiles. The training data 304 of the computing device 302 can be populated by one or more data sources 306 (e.g., data source 1, data source 2, data source n, etc.) over a period of time (e.g., t, t+1, t+n, etc.). In some examples, the training data can be one or more DNA methylation profiles without a label to be used in unsupervised clustering. In other examples, training data 304 can be labeled data (e.g., one or more tags associated with the data). For example, training data can be one or more DNA methylation profiles and a label (e.g., tumor classification, sub-classification, etc.) can be associated with each DNA methylation profile. The computing device(s) 302 can continue to receive data from the one or more data sources 306 until the neural network 308 (e.g., convolution neural networks, deep convolution neural networks, artificial neural networks, learning algorithms, etc.) of the computing device(s) 302 are trained (e.g., have had sufficient unbiased data to respond to new incoming data requests and provided an autonomous or near autonomous tumor classification). In some examples, the neural network can be a convolutional neural network, for example, utilizing five layer blocks, including convolutional blocks, convolutional layers, and fully connected layers. While example neural networks are realized, neural network 308 can be one or more neural networks of various types are not specifically limited to a single type of neural network or learning algorithm. In other examples, a cluster model can be generated (e.g., group correlated features such that specific features are used for each classification). In these instances, features with non-zero coefficients are used in a cluster model.

In some examples, while not shown here, the training data 304 can be checked for biases, for example, by checking the data source 306 (and corresponding user input) verse previously known unbiased data. Other techniques for checking data biases are also realized. The data sources can be any of the sources of data for providing the input DNA methylation profiles as described above in this disclosure.

The computing device(s) 302 can receive user (e.g., physician) input 310 related to the data source. In some examples, the user input 310 and the data source 306 can be temporally related (e.g., by time t, t+1, t+n, etc.). That is, the user input 310 and the data source 306 can be synchronous in that the user input 310 corresponds and supplements the data source 306 in a manner of supervised or reinforced learning. For example, a data source 506 can provide a methylation profile at time t and corresponding user input 510 can be tumor family, class, sub-class, or patient demographics at time t. While time t may actually be different in real-world time, they may be synchronized in time with respect to the data provided to the training data. In other examples, the data source 306 may be used in a manner of unsupervised learning without user input.

The training data 304 can be used to train a neural network 308 or learning algorithms (e.g., convolutional neural network, artificial neural network, etc.). The neural network 308 can be trained, over a period of time, to automatically (e.g., autonomously) determine what the user input 310 would be, based only on received data 312 (e.g., DNA data, methylation profiles, etc.). For example, by receiving a plurality of unbiased data and/or corresponding user input for a long enough period of time, the neural network will then be able to determine what the user input would be when provided with only the data. For example, a trained neural network 308 will be able to receive a methylation profile of a sample tumor (e.g., 312) and based on the methylation profile determine the classification of the tumor that a physician would manually identify (and that could have been provided as user input 310 during training). In some examples, this can be based on families, classes, and sub-classes identified using unsupervised learning. In other examples, this can be based on labels associated with the data as described above. The output from the trained neural network can be provided to a cluster model 314 for treating a patient. In some examples, the output from the trained neural network can be inputted directly into a cluster model 314 to predict a classification and/or subclassification of the tumor.

Trained neural network system 316 can include a trained neural network 308, received data 312, and cluster model 314. The received data 312 can be information related to a patient, as previously described above. The received data 312 can be used as input to the trained neural network 308. Trained neural network 308 can then, based on the received data 312, classify the received data and/or determine a recommended course of action for treating the patient, based on how the neural network was trained (as described above). The recommended course of action or output of trained neural network 308 can be used as an input into a cluster model 314 (e.g., to predict the classification of the tumor of the patient to which the received data 312 corresponds). In other instances, the output from the trained neural network can be provided in a human readable form, for example, to be reviewed by a physician to determine a course of action.

For clarity of explanation, in some instances the present technology may be presented as including individual functional blocks including functional blocks comprising devices, device components, steps or routines in a method embodied in software, or combinations of hardware and software.

In some embodiments the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the above-described examples can be implemented using computer-executable instructions that are stored or otherwise available from computer readable media. Such instructions can comprise, for example, instructions and data which cause or otherwise configure a general purpose computer, special purpose computer, or special purpose processing device to perform a certain function or group of functions. Portions of computer resources used can be accessible over a network. The computer executable instructions may be, for example, binaries, intermediate format instructions such as assembly language, firmware, or source code. Examples of computer-readable media that may be used to store instructions, information used, and/or information created during methods according to described examples include magnetic or optical disks, flash memory, USB devices provided with non-volatile memory, networked storage devices, and so on.

Devices implementing methods according to these disclosures can comprise hardware, firmware and/or software, and can take any of a variety of form factors. Typical examples of such form factors include laptops, smart phones, small form factor personal computers, personal digital assistants, rackmount devices, standalone devices, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

### EXAMPLES

### Example 1: DNA Methylation-Based Classification of Common Renal Tumors

The 2022 WHO classification of renal tumors contains over 40 benign and malignant renal neoplasms. The heterogeneity of these entities is well documented and is reflected in their variable histology, molecular characteristics, and clinical behavior. The current standard of care for renal neoplasm diagnosis is a review of the H&E, immunohistochemistry, and, in some cases, genetic testing. However, this process is subject to inter-observer discordance among pathologists with potentially significant consequences for the patient. Especially challenging renal neoplasms include microscopically similar entities such as hybrid oncocytic chromophobe tumors (HOCT), chromophobe renal cell carcinomas (ChRCCs), benign oncocytomas, and various eosinophilic entities that are poorly defined. DNA methylation profiles of neoplasms represent a combination of signatures derived from the cell of origin and the epigenetic changes associated with tumorigenesis. Array-based genome wide methylation profiling is robust, reproducible, and can be performed on formalin fixed paraffin embedded tissues which is the most common tissue preparation in clinical pathology laboratories. Recent studies on neoplasms of the central nervous system and soft tissue and bone have demonstrated that the neoplasm DNA methylation signals are so specific that they can be used to construct clinical-grade DNA methylation-based classifiers. Extensive experience with central nervous system tumors has shown the value of methylation profiling to identify clinically relevant entities.

The methylation status of limited numbers of methylation sites has been shown to distinguish major RCC histologies. To create an initial version of an expansive DNA methylation-based renal neoplasm classifier private and publicly available data were used to identify renal neoplasms with distinct DNA methylation profiles and to train a prospective classifier. Although both epithelial and non-epithelial neoplasms were included in this study, the analysis focused on the three major types of epithelial renal neoplasms: clear, papillary, and eosinophilic entities.

### Study Schema and Development of Reference Set of Kidney Neoplasms

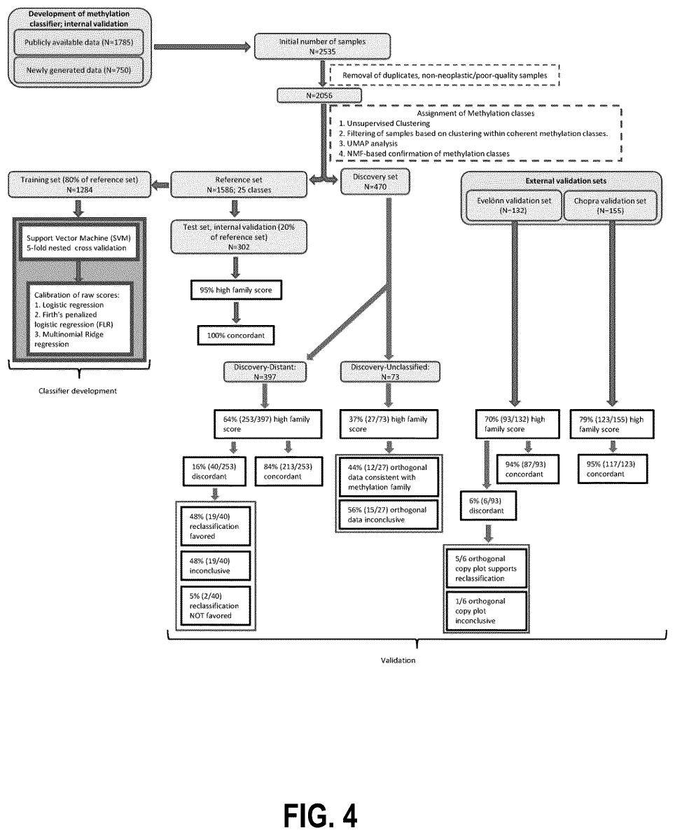

The study schema (FIG. 4) describes the curation approach for available and newly generated methylation data on renal neoplasms for the development and validation of a classifier. Briefly, taking publicly available methylation data (from either the 450 k or EPIC/850 k array) and newly generated data (EPIC/850 k array) 2531 samples were evaluated. After removal of duplicates, specimens with failed quality control metrics, and specimens not relevant to the classes examined, 2086 samples were used for development and internal testing of a classifier. One pair of tested specimens originated from 2 distinct tumors in the same patient. Methylation data were analyzed with unsupervised methods including hierarchical clustering and Uniform Manifold Approximation and Projection for Dimension Reduction (UMAP) to identify differentially methylated clusters. Based on a previously suggested practice, a minimum of 7 cases per cluster were required to define a class.

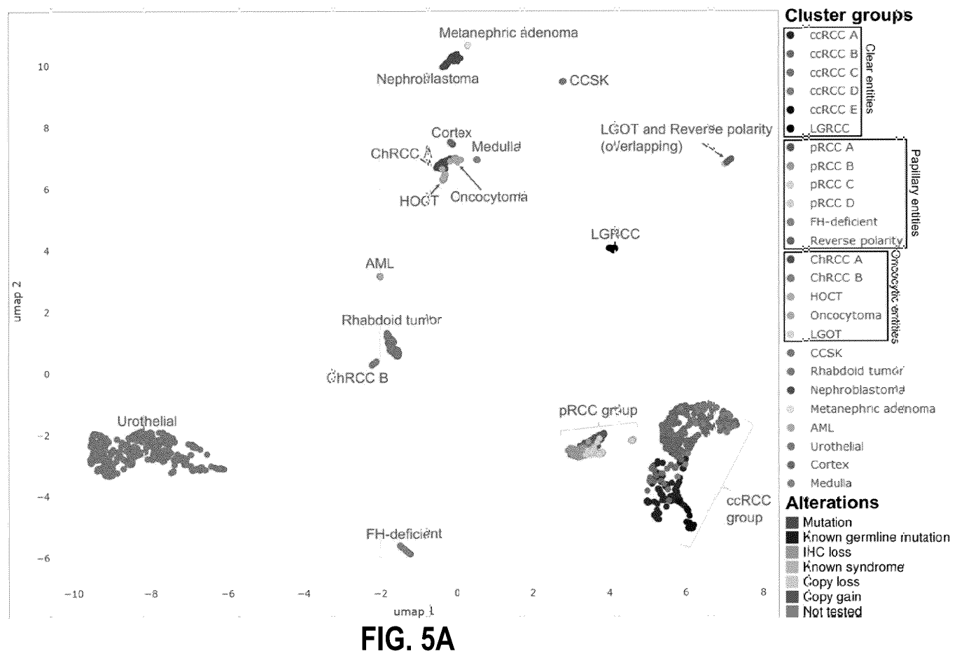

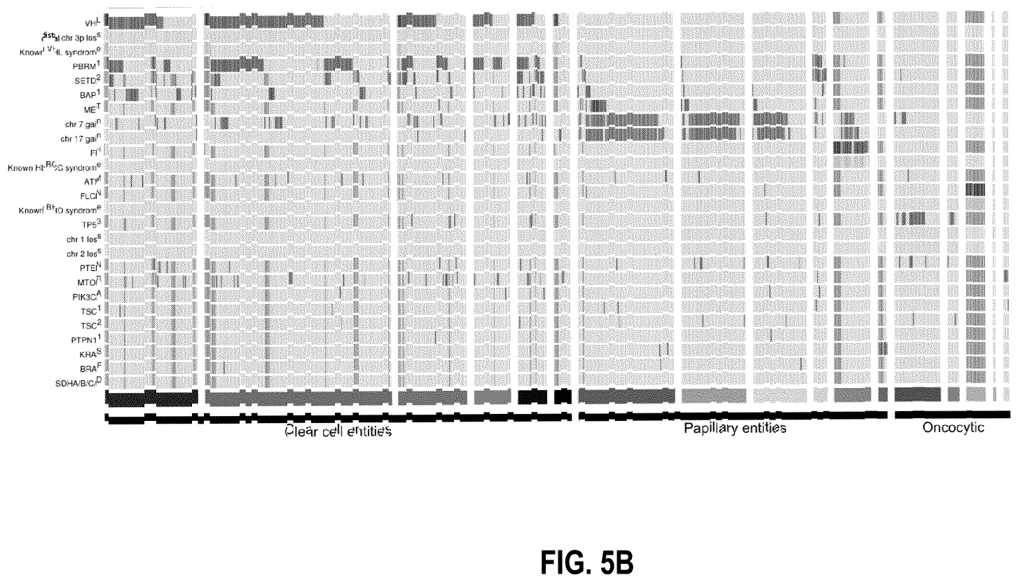

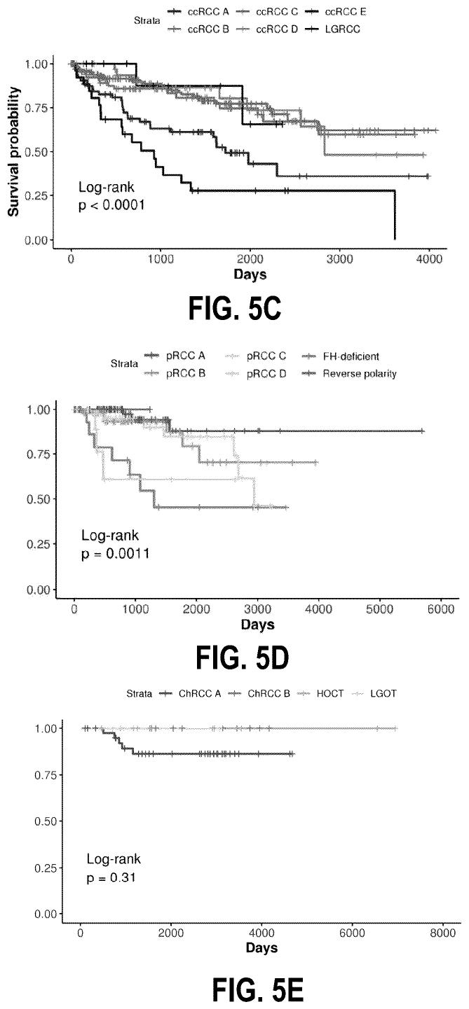

After limiting samples in the reference set to a well-annotated set that formed coherent groups, the reference set, which was separated into a training classifier set and internal classifier test set, included 1594 samples. A separate set of 492 samples were used as a “Discovery” set (FIG. 4). Technical and biologic factors (array type, sex, age, source, and frozen tissue versus formalin-fixed paraffin embedded tissues) were addressed separately to exclude a confounder effect. Unsupervised analyses resulted in a total of 16 families and 25 methylation classes, including 23 neoplasm classes as well as 2 non-neoplasm classes (renal cortex and medulla). The 23 neoplastic methylation classes corresponded to 13 distinct WHO entities of renal neoplasms and 1 class of urothelial neoplasm (FIG. 5A). Among the 13 distinct WHO-defined renal neoplasms, 10 had a 1:1 relationship of WHO tumor type to methylation class, while the remaining 3 WHO entities diverged into multiple classes, including ccRCC (5 classes), pRCC (4 classes) and ChRCC (2 classes) FIG. 5A). The 5 ccRCC and 4 pRCC classes constitute the ccRCC and pRCC family respectively. The two ChRCC classes (A and B) were considered distinct entities and are therefore not parts of a family.

Clear Cell, Papillary, and Eosinophilic Renal Neoplasms Form Multiple Methylation Clusters which Correspond to Morphologically, Genomically, and Clinically Distinct Entities

Histologically classic ccRCCs formed a large continuous cluster which was subdivided into 5 partially overlapping clusters (ccRCC A-E) (FIG. 5A). Patients with ccRCC A and B were younger compared to those with ccRCC C-E with the oldest patients found in cluster ccRCC E. Consistent with prior literature, most cases in all 5 ccRCC clusters had VHL alterations and distal chromosome 3p loss. A small subset of ccRCC A-C had known VHL syndrome. SETD2 was mutated in about half of ccRCC E but only up to 18% of ccRCC A-D. BAP1 was mutated in 26% and 28% of ccRCC A and E respectively but only in up to 7% of ccRCC B-D. Six to 13% of ccRCC A-E had mTOR mutations (FIG. 5B). ccRCC A and E had a significantly higher grade and pathologic stage and a significantly poorer overall survival (FIG. 5C).

A cluster of low-grade clear cell neoplasms with frequent papillary features was named “low grade RCC” (LGRCC). Three of 14 cases had germline VHL mutations but no somatic VHL mutations or chromosome 3p loss. Two of 11 sequenced cases had activating MTOR mutations (S2215Y and I1973F) (FIG. 5B). Recurring copy number changes were not identified although 10 of the 25 cases with copy number data had a flat profile.

pRCC A-C formed a large continuous cluster with partially overlapping clusters. pRCC D was distinct but in close proximity to pRCC A-C throughout the UMAP iterations. Neoplasms in pRCC A-D were found in progressively older patients with the oldest patients found in pRCC D. pRCC A-C had a largely overlapping molecular profile with a majority of cases having gain of chromosome 7 (63.8-82.1%) and 17 (57.4-80.4%). Eleven of 12 pRCC D had a mutation in PBRM1 or SETD2. pRCC D was also the only pRCC class with simultaneous mutations in PBRM1 and SETD2 (in 5/12 cases) and had a relatively low frequency of gain in chromosome 7 (25%) and 17 (8.3%). MET was mutated in 6-20% of cases within the pRCC clusters (FIG. 5B). Pathologic stage progressively increased from pRCC A to D and pRCC D had the poorest prognosis of all pRCC clusters (FIG. 5D).

Patients with neoplasms in the FH-deficient cluster were the youngest of all patients in the papillary clusters. These neoplasms also had higher pathologic stage, and a very poor prognosis compared to the other papillary clusters. Most cases had germline FH mutations (FIG. 5B). One notable case was case 1660 which had no FH mutation but did have a deep FH deletion and a CAPZA2::MET fusion (these alterations were not surfaced in the oncoplot). Another 4 cases in the FH-deficient cluster did not have FH-related changes. These cases are: 1662 which has a RPL11::TCEB3 fusion and SLC1A3 deep deletion, 84 with an NF2 deep deletion, 592 with a PTPRD deep deletion, and 591 with a SRRM3 deep deletion. It is not clear whether these alterations are directly related to the phenotype of these FH-wild type cases. The cluster which consisted of eosinophilic papillary neoplasms overlapped with LGOT (see below) and, with one exception, had activating KRAS mutations, low stage disease, and an excellent overall survival (FIG. 5D). This cluster corresponds to the recently described pRCC subset referred to as “PRCC with reverse polarity”. The single KRAS wild-type case (case 200) had classic morphologic reverse polarity features and had been sequenced only for KRAS hotspot mutations.

The oncocytic clusters consisted of chromophobe A and B, HOCT, oncocytoma, and LGOT clusters. A tendency towards overlap was identified between multiple oncocytic clusters, most prominently ChRCC A, oncocytoma, and HOCT (FIG. 5A).

ChRCC clusters A and B had distinct DNA methylation profiles but similar morphology, age distribution, DNA mutations, DNA copy number changes, stage, grade, and survival characteristics. Hybrid tumors formed their own cluster and all cases either had a germline FLCN mutation or known history of BHD (FIGS. 5A and 5B).

The 17 oncocytoma patients were the oldest in all the oncocytic clusters and had chromosome 1 loss in 12 cases. The 9 low-grade oncocytic tumor (LGOT) cases were also found in relatively older patients, harbored MTOR mutations in 3 cases, a TSC1 mutation in one case (mutually exclusive with the MTOR mutations), and a chromosome 1 loss in one case (mutually exclusive with the MTOR mutations and TSC1 mutation). All cases with available data were of pathologic Stage I. Copy changes were non-specific although of the 9 cases, 5 had a flat copy number profile. All oncocytic clusters had an excellent prognosis although ChRCC A had a trend towards poorer overall survival (FIG. 5E).

### Non-Neoplastic Kidney and Additional Neoplasms Form Distinct Clusters

Additional clusters included distinct kidney cortex and medulla clusters, and clusters of various neoplasms: CCSK, rhabdoid tumor, nephroblastoma, metanephric adenoma, angiomyolipoma, and urothelial cancers (of both upper and lower GU tract) (FIG. 5A).

CD14 by Cibersort Correlates with Prognosis in ccRCC Classes

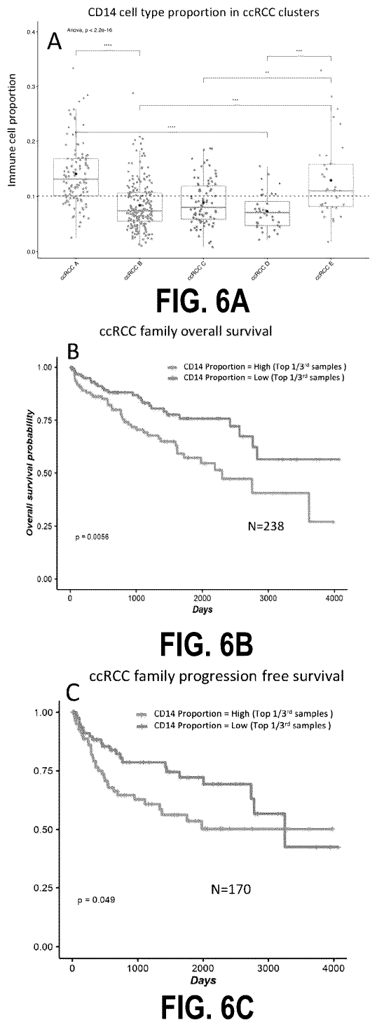

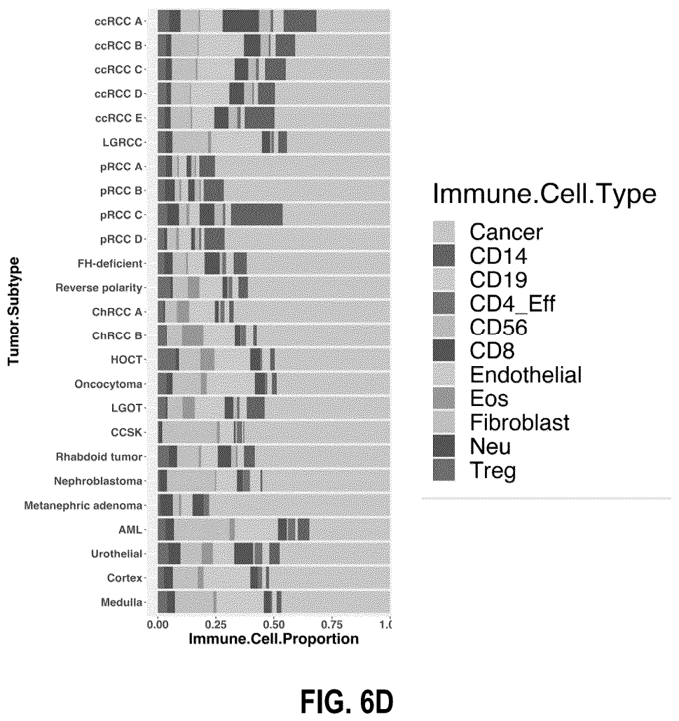

Deconvolution of methylation data into immune cell abundance by Cibersort revealed various inter-cluster differences which were most pronounced in the clear cell and papillary groups. ccRCC A and E had a relatively high CD14 fraction (FIG. 6A). The ccRCC group with the lowest third proportion of CD14 abundance by Cibersort had a significantly better progression free survival and overall survival compared to the highest third (FIGS. 6B and 6C). Other prominent overrepresentations included CD14 in pRCC C, and CD8 in ccRCC A and pRCC C (FIG. 6D).

### Relationships of Methylation-Based Tumor Classes Compared to Expert Pathology Review of the H&E Slide

Initial consensus between GU pathologists was achieved in 102/140 cases. For 89 of these, the consensus diagnosis was concordant with the methylation class. For the remaining 13 the consensus diagnosis and methylation class did not coincide, and additional molecular details were sent to the GU pathologists for re-evaluation. Six of these 13 cases were re-diagnosed as consistent with the methylation class but the remaining 7 could not be reconciled with the methylation class. Of these 7, one clustered in the LGOT and 6 in the LGRCC cluster.

Initial histological consensus was not achieved in 38 cases. After molecular details were sent, 28 cases were rediagnosed as consistent with the methylation class but the remaining 10 could not be reconciled with the methylation class. Three of these clustered in the LGOT and 7 in the LGRCC cluster.

The LGOT cluster had a wide range of morphology-based diagnoses by the GU pathologist including ChRCC, oncocytoma, ccRCC, oncocytic tumor, LOT, PRCC, SDH RCC, and “unclassified” (data not shown).

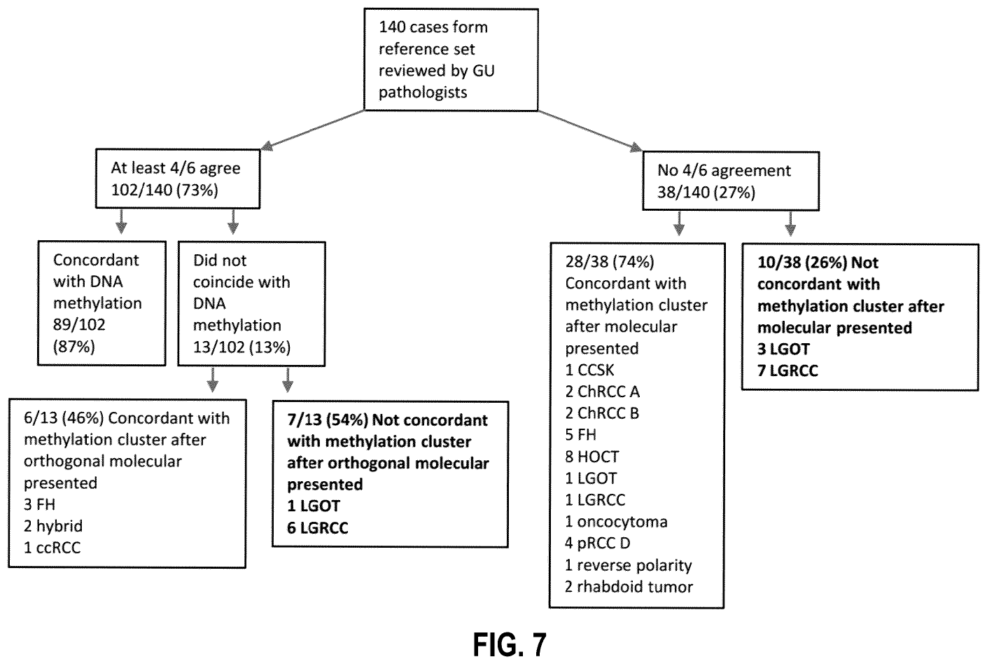

Overall, the consensus between methylation class and pathologist impression was 88% (123/140) cases. Of the 140 reviewed cases, a total of 4 LGOT and 13 LGRCC cluster cases emerged as particularly difficult to diagnostically reconcile even after molecular profiles were provided (FIG. 7).

### Classifier Development and Internal Validation

The 1586-sample reference set was separated by an 80-20 split and a classifier was trained on 80% (n=1291) of the samples using a support vector machine (SVM) model with 5-fold nested cross-validation. Raw and calibrated scores were calculated as part of the classifier output. To assess an internal performance metric, cross-validation was conducted with an estimated error rate of 0.07 for raw scores and a discriminating power of 0.9981% by area under receiver operating characteristic curve analysis. SVM-MR (Support vector machine-Multinomial regression) model was selected with the highest AUC (0.998274) and minimum mLogloss (0.2300) as the final model. The classifier was tested on the remaining 20% (n=302) samples in the reference set. To distinguish high versus low confidence matches, a threshold of 0.84 was chosen.

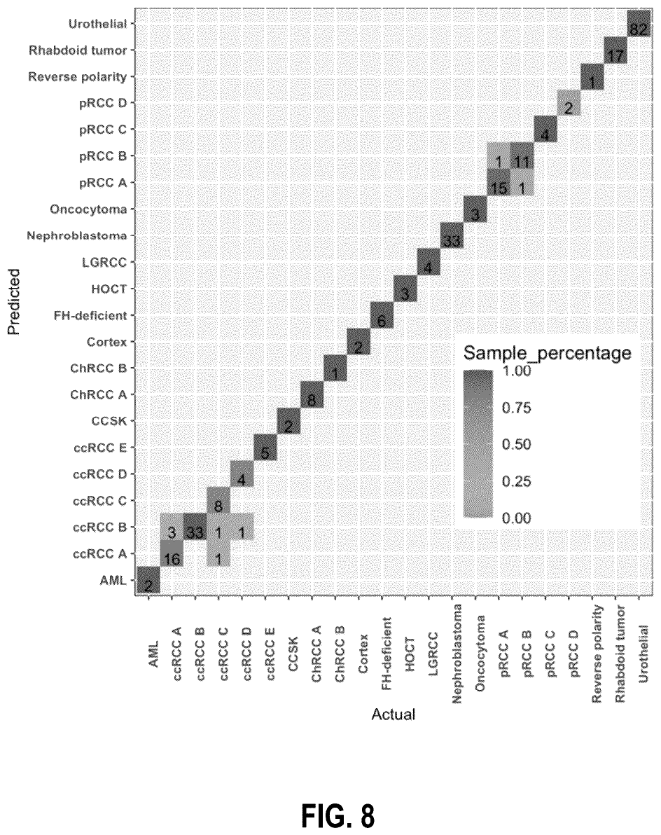

Medulla and LGOT were not included in this 302-sample test set due the small number of specimens in these clusters. Ninety five percent of cases (287/302) had a high family score and 100% of these had an initial UMAP cluster assignment concordant with that of the methylation family classification. Eighty nine percent (270/302) of cases had a high class score and 97% of these (262/270) had an initial UMAP cluster assignment concordant with that of the methylation class classification. All class discrepancies were due to class misclassification within the ccRCC or pRCC families (e.g., ccRCC A to ccRCC B, etc.) (FIG. 8).

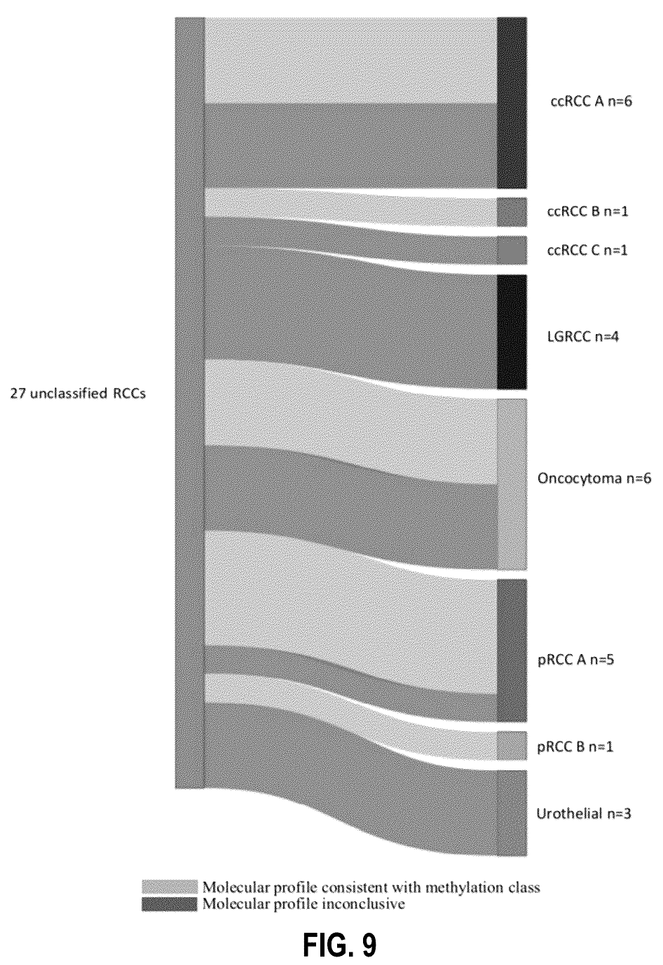

The classifier was applied to a set of samples that were not included in the reference set (Discovery set, n=470). This set consisted of samples that were excluded from the reference set based on their excessive distance from a coherent cluster (Discovery-Distant, n=397) or were histologically unclassified (Discovery-Unclassified, n=73). When the classifier was applied to the 73 Discovery-Unclassified samples, 27 cases (37%) received high family and class scores. Orthogonal molecular data (copy plots in all cases and sequencing data when available) was examined that could help determine if the methylation classification could be supported. Twelve of the 27 cases (44%) had orthogonal molecular data that were supportive of the methylation family and class. For the remaining cases, the available orthogonal data were not helpful in confirming the methylation class (FIG. 9). For example, one unclassified case matched to the pRCC family (pRCC A), had complex copy number changes in addition to a reported PPARG-ITGB5 fusion (case 1649). In a second example, an unclassified case matched to the ccRCC family (ccRCC A), had a complex copy number profile, and a SETD2 mutation (case 1786). Neither case had a morphology characteristic of the assigned methylation family or class.

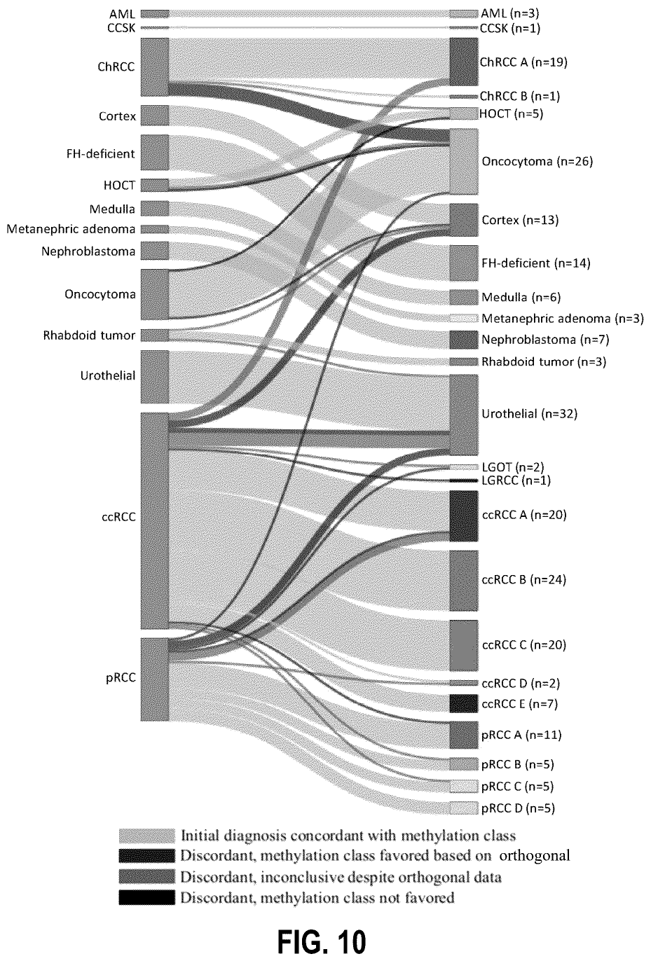

Among the 253/397 (64%) cases in the Discovery-Distant set that received a high family score, 213/253 (84%) had a concordant initial diagnosis and methylation family. The H&Es (if available) and all other available orthogonal data were reviewed for these 40 cases. Of these, the methylation family was favored in 19 cases, not favored in 2 (cases 1943, 2008), and inconclusive in 19 (FIG. 10).

Two hundred and thirty-five Discovery-Distant had a high class score, all of which also had a high family score. The 18 cases which had a high family score, but low class score were all class level misclassifications within the ccRCC or pRCC families.

### External Validation of Classifier

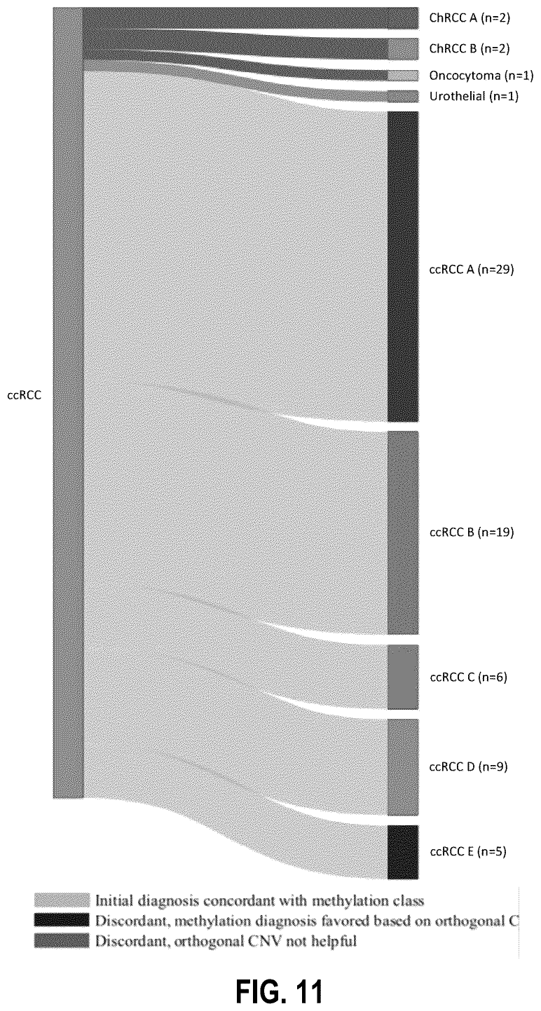

Two external datasets were obtained to test the classifier. Idat pairs from 132 non-metastatic, histologically diagnosed ccRCC were examined (“Evelönn” dataset, H&E images were not available for review but copy plots were generated and served as orthogonal data). Ninety three of 132 (70%) received a high family score. Of these, 87 (94%) matched to ccRCC and the remaining 6 received a classifier label other than ccRCC: 2 ChRCC A, 2 ChRCC B, 1 oncocytoma, and 1 urothelial. The 4 ChRCC and 1 oncocytoma classifier-labeled cases had copy plots consistent with the methylation classifier-assigned label. The 1 urothelial classifier-labelled case had a non-specific flat copy plot and was therefore not conclusive. Overall, after considering the orthogonal copy plot data, 92 of the 93 (99%) cases with a high family score were considered accurately classified. Seventy four of 132 cases (56%) received a high specific class. Ninety two percent of these were consistent with the initial diagnosis. The 6 discordant cases were the same ones as the discordant cases with a high family score (FIG. 4, FIG. 11).

Idat pairs from 155 renal neoplasms of various histological diagnoses were then examined (“Chopra” dataset, H&E images were not available for review and copy plots could not be generated).

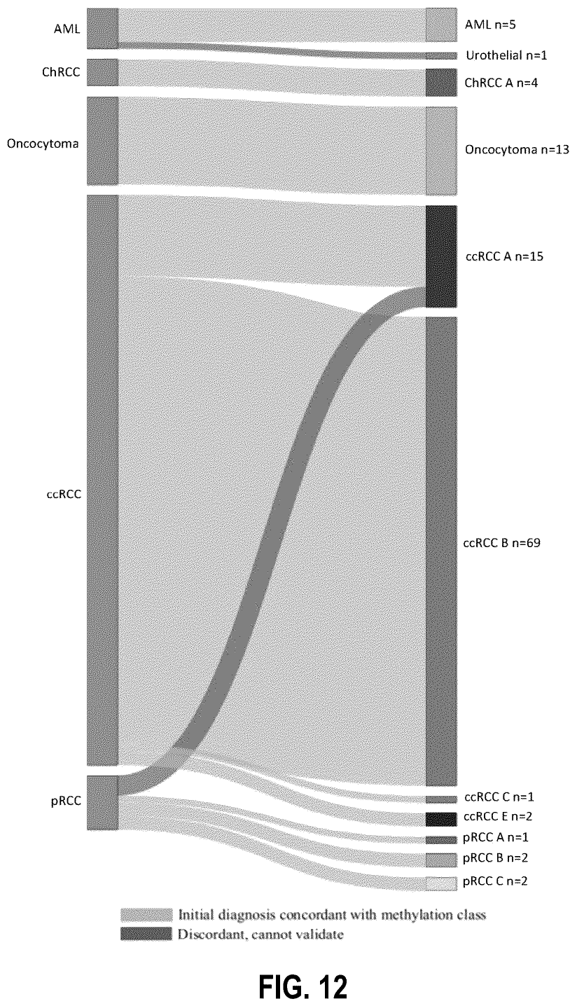

One hundred and twenty three of 155 cases (79%) received a high family score. Of these 123, 117 (95%) matched to a family that was concordant with the provided histological diagnosis. Among the 6 discordant cases, 3 cases initially diagnosed as pRCC were classified as ccRCC, 1 oncocytoma as ccRCC, 1 AML as ccRCC, and 1 AML as urothelial carcinoma. One hundred and fifteen of 155 cases (74%) received a high class score. Ninety seven percent of these were consistent with the initial diagnosis. The 4 discordant cases were within the 6 which had a high family score and were discordant. Further confirmation of the discordant cases was not possible due to a lack of orthogonal data (FIG. 4, FIG. 12).

Overall, 216/287 (75%) external cases (Evelonn and Chopra datasets) received a high family score. Ninety-seven percent of these were accurately classified.

### Data Collection

One objective was to collect enough DNA methylation profiles of diverse tumor types in renal cancer. So, a collection of newly processed samples was used as well as publicly available DNA methylation data to have a variety of diverse kidney tumors from various sources. A subset of samples (n=89) were profiled as a part of routine clinical methylation testing for kidney cancer at NIH. One subset of samples (n=75) was collected from collaborators at UCSF and another subset of samples (n=55) from NYU. All samples were profiled in accordance with standard manufacturer's protocol for Illumina EPIC array (http://www.illumina.com/). In the final reference dataset, a majority of samples were downloaded from different publicly available data sources such as: CGR (n=14), Clinical Proteomic Tumor Analysis Consortium (https://portal.gdc.cancer.gov/projects/CPTAC-3) (n=90), Therapeutically Applicable Research to Generate Effective Treatments-TARGET (https://ocg.cancer.gov/programs/target) (n=137), TCGA (https://www.cancer.gov/) (n=895), PMID: 26515236 (n=27) [2], PMID: 30131446 (n=18) [3], PMID: 31115548 (n=7) [4], PMID: 31426508 (n=3) [5], PMID: 31708418 (n=84) [6], PMID: 31862972 (n=35) [7], PMID: 32859926 (n=10) [8], PMID: 33015531 (n=9) [9], PMID: 33397444 (n=35) [10], PMID: 33414138 (n=7). In the final dataset, 1586 samples were collected with a combination of Illumina 450 k and Illumina EPIC array and 25 class labels: ccRCC A, ccRCC B, ccRCC C, ccRCC D, ccRCC E, LGRCC, pRCC A, pRCC B, pRCC C, pRCC D, FH-deficient, Reverse polarity, ChRCC A, ChRCC B, HOCT, Oncocytoma, LGOT, Urothelial, CCSK, Metanephric adenoma, Rhabdoid tumor, AML, Nephroblastoma, Cortex, and Medulla.

Kidney cases with fusions were excluded from the reference set. All cases with fusions were identified in PMID:29617662, 26536169, 23792563. The excluded fusions contained the following partners: TFE3, TFEB, ALK, MET, TACC3, TCEB3, and HNF1B.

### Prescreening of Idat Files as Quality Check

In the dataset, significant number of samples were collected from publicly available data with possibilities of having bad quality samples, so multiple methods were used to check the quality of idat files. In a first step, the mean detection p-value was calculated for each sample to assess quality of the samples in terms of the overall signal consistency. A bad quality sample showed large mean detection p-values as compared to a good sample. All samples with more than 0.05 mean detection p-values were removed. Additionally, each idat file was screened for median log (base 2) of methylated and unmethylated intensities and samples with median intensity below cutoff 9 were removed from the analysis.

### Data Preprocessing

Raw intensity data files (IDATs) for both 450 K and EPIC arrays were combined into a matrix with a common probe set (452,453 probes). All samples were processed and normalized by single sample noob function provided in the Minfi R package. Correction in the data for array type (450 k/EPIC) was implemented by fitting univariate, linear models to the log 2-transformed intensity values by using “removeBatchEffect” function in limma package. Methylated and unmethylated signals were corrected separately as described in a previous study. After correcting individual methylated and unmethylated signals, Beta-values were calculated from the corrected intensities using an offset of 100.

In the next step, a set of probes were removed, which consists of probes on X and Y chromosomes, probes related with SNPs (single nucleotide polymorphism), probes not uniquely mapped to human reference genomes and probes not included on EPIC array. After filtering of probes, 357483 common probes were selected on EPIC/450 k array for further analysis. Many publicly available datasets were used, so it was a prerequisite to see any possible batch effect in any of the data subset. 17 batches were created for all 1586 samples based on their source. BEclear R package was used to detect batch effect in our final beta matrix with variable size of top 200 K, 100 K, and 50 K most variable probes. A minimum of 5 samples (as recommended in the BEclear R package) with p-value <0.001 were considered for possible batch effect. Significant batch effect was not found in any of the data subsets.

### Unsupervised Clusterin

A range of most variable probes (10 k, 20 k, 25 k, 30 k, 50 k) were used and multiple UMAPs were generated to find maximum segregation between clusters and finally selected the 20 k most variable probes for UMAP generation. Prior to UMAP generation we calculated principal components (n=25) using singular value decomposition (SVD) in the RSpectra package. We used the first 25 principal components (PC) as input to the UMAP function (n_neighbors=10, n_components=4, metric=“cosine”, min_dist=0, spread=1) of the uwot R package. All other parameters were set at their default values.

### Classifier Development

The top 10 k most variable probes were used as input features for classifier development. All 1586 samples were randomly assigned to training set (1284) and primary test set (302) by keeping an approximate 0.8 and 0.2 proportion respectively. The SVM (support vector machine) was selected, and a 5-fold nested cross validation was used to develop the classifier. First, the whole training dataset was randomly divided into 5 subsets. For each of the outer CV loops one of these sets was used as a test while the other four were merged as training. In each training set the samples were further divided into 5 subsets to generate 5 inner CV loops similarly. Hence a total of 5 outer loops were made with each derived 5 inner ones. The outer loops were used to evaluate the generalization of the model while the inner ones were used to avoid over-fitting during the calibration step (the test sets of the inner loops were used as the input of the calibration model, rather than the inner or outer training sets). The mean error (ME) of the classifier was calculated, which is defined as the proportion of incorrectly classified cases over all classes divided by the total number of cases. Three different calibration methods were compared to get calibrated scores (i) Platt scaling, implemented by fitting LR (ii) Firth's penalized likelihood LR (FLR) and (iii) ridge-penalized multinomial logistic regression (MR). After evaluating the mean error (ME), Brier score (BS), mLogloss and AUC (area under curve), multinomial ridge regression (MR) was selected as the best calibration method with an error of 0.062.

Two distinct classifications with corresponding scores were produced for each specimen: a specific methylation class and a family class. The difference between these was final degree of granularity for ccRCC and pRCC outputs: specific methylation class had distinct ccRCC A-E and pRCC A-D outputs and scores. On the other hand, all ccRCCs and pRCCs were also considered as single respective family classes. Due to their UMAP distance, ChRCC A and ChRCC B were considered distinct families. Family scores were calculated by adding the calibrate scores of all classes within that family.

### Oncoplot Generation and Other Plots

The oncoplot was generated using the ComplexHeatmap R package. Somatic and germline mutations were included. The genes that were included are: VHL, PBRM1, SETD2, BAP1, MET, FH, ARID1A, KDM5C, KDM6A, NF1, NF2, STAG2, DNMT3A, FGFR3, SMARCB1, ATM, FLCN, TP53, PTEN, MTOR, PIK3CA, TSC1, TSC2, PTPN11, KRAS, BRAF, and SDHA/B/C/D. Known clinical history of VHL, HLRCC, or BHD was also incorporated. Cases were excluded from the oncoplot if they did not have any sequencing data (somatic or germline) or confirmed clinical syndrome history.

Boxplots, bargraphs, and survival plots were created using ggboxplot, ggplot, and ggsurvplot R packages.

### External Validation

Two external validation datasets were used: Chopra validation set (155 samples, beta values downloaded from: https://osf.io/y8bh2) and Evelönn set (132 samples from 121 patients). Missing beta values were imputed using the BEclear R package.

### Copy Number Analysis

Raw intensities of methylated and unmethylated signals were imported from idat files using the minfi package's “preprocessRaw” function. Medulla and cortex samples (n=24) were used as normal references. CNV analysis and segment means were calculated using the “conumee” R package. All parameters such as detail and excluded regions were set to their default values as provided in the package. Once segment values were obtained from the Conumee package, the “GenVisR” R package was used to create a copy number frequency plot for each cluster. A threshold was set for segments value of ±0.1 for any loss or gain.

### Immune Cell Fraction Estimation

DNA methylation deconvolution was used to estimate immune cell fraction in each of the tumor classes. The kidney signature matrix available in the “MethylCIBERSORT” R package was used to create input files for CIBERSORT. CIBERSORTx was used for fraction estimation. All parameters were set at their default values.

### GU Pathologist Review

Six expert genitourinary (GU) pathologists reviewed a subset of reference cases which, based on their UMAP cluster location, were considered to be members of distinct clusters. One hundred and forty reference set cases were selected for review. We enriched this morphology review for cases in the LGOT cluster (6 of the 9 reference LGOT cases were reviewed), and LGRCC cluster (19 of the 25 reference LGRCC cases were reviewed). Agreement was defined as diagnosis of the same WHO class by at least 4/6 GU pathologists, and anything less was defined as disagreement. For all cases with disagreement or cases with agreement that did not coincide with the DNA methylation cluster, all available molecular data (copy changes at a minimum) were provided to the GU pathologists and a new diagnosis was requested. Agreement by at least 4/6 GU pathologists was again required for agreement.

### Discussion

The identification of neoplasm DNA methylation clusters can be used to create classifiers that complement histology. This example focuses on the development of a classifier for epithelial renal neoplasms that broadly fall under clear, papillary, and eosinophilic groups. These clusters segregate into morphologically, molecularly, and clinically distinct entities.

Among clear cell entities, ccRCC classes A and E encompass the majority of high grade, high stage, and poor prognosis ccRCCs. The overrepresentation of SETD2 mutations in ccRCC E and BAP1 mutations in ccRCC A and E is consistent with previous data although these mutations were not specific to the respective clusters and therefore are unlikely to be clinically useful. The presence of cases with germline VHL mutations and/or clinical history of VHL in ccRCC A-C suggests that ccRCCs in the context of VHL syndrome do not have a strongly distinct methylation profile compared to sporadic cases (FIG. 5A).

The high CD14 fraction in ccRCC A and E is suggestive of the presence of monocytic cells which have been previously shown to be an independent poor prognostic factor in ccRCC due to the immunosuppressive effect associated with monocytic cells (FIG. 6A). The presence of a high CD8 fraction in ccRCC A is indicative of immune activation (FIG. 6D). This raises the question whether ccRCC A might be a good immunotherapy candidate although CD8 infiltration is not known to be a predictor of immunotherapy response in ccRCC.

Cases that overlap the morphologic and molecular definition of ccPRCC, i.e., lack of chromosome 3p loss and VHL somatic mutations, were found in the LGRCC cluster. A subset of these LGRCC cases occurred in the context of VHL syndrome which is also well-described in ccPRCC (FIGS. 5A and 5B). The lack of distinct somatic molecular findings and the wide differential diagnosis of this cluster is reflected by the frequent difficulty of GU pathologists to diagnose these cases (FIG. 7).

In addition to cases with “classic” ccPRCC morphology, the LGRCC cluster also contained RCCs with leiomyomatous stroma. Cases of RCC with papillary clear cell morphology, MTOR mutations, and prominent leiomyomatous stroma have been previously described as constituting a distinct class of RCC. LGRCC's significantly lower nuclear grade compared to other clear cell entities is consistent with its relatively low clinical aggression (FIG. 9). However, it is worth noting that two reference LGRCC cases were in deceased patients (cases 253, 604). One of these cases had high stage and high grade disease (case 604). Without being limited to any one theory, RCCs with leiomyomatous stroma and ccPRCCs may be a part of the LGRCC continuum which include a subset of clinically aggressive cases.

pRCC D is highly enriched for PBRM1 and SETD2 mutations and has the poorest prognosis of all pRCC clusters. Although PBRM1 and SETD2 mutation cooccurrence was not found in every case of pRCC D, the cooccurrence was unique to this group. Without being limited to any one theory, the PBRM1-related SWI/SNF complex and SETD2-related chromatin modifier pathway are both activated in pRCC D, with the downstream consequences of this compound activation contributing to the dismal prognosis of pRCC D.

The immune cibersort profile of pRCC C is similar to that of ccRCC A in that it also has a high proportion of CD14 and CD8. However, in contrast to ccRCC A, pRCC C has a relatively favorable prognosis (FIG. 5C). This difference likely indicates that the aggressiveness of these neoplasms is also dependent on factors other than easily defined immune subsets.

The eosinophilic groups ChRCC A, HOCT, and oncocytoma are so close on the UMAP that they partially overlap (FIG. 5A). On the other hand, despite the highly distinct and distant ChRCC A and ChRCC B clusters, the only appreciable difference between the two was a non-significant trend towards relatively worse overall survival in ChRCC A (FIG. 5C). However, without analysis of a larger cohort it is difficult to say whether methylation group UMAP proximity or distance indicates true “biological” relationships.

It is not clear whether LGOT constitutes a previously described entity. The presence of MTOR or TSC1 mutations in 4/6 sequenced LGOT neoplasms suggests that MTOR pathway mutations may be a characteristic of this entity. The recent GU WHO mentions various newly described clinically indolent eosinophilic neoplasms such as eosinophilic, solid and cystic (ESC) RCC, high grade oncocytic renal tumors (HOT), eosinophilic vacuolated tumor (EVT), and low-grade oncocytic tumor of the kidney (LOT). Morphology review by expert GU pathologists suggests that the LGOT cluster is most consistent with LOT although the GU pathologist's difficulty to come to a consensus (at least as LOT) is likely a consequence of morphology which largely overlaps with that of other entities and of the lack of specific somatic alterations. DNA methylation analysis of a cohort ESC, HOT, EVT, LOT, and other eosinophilic neoplasms may help consolidate some of these entities.

The validation set classifier score decreased sequentially when the classifier was applied to the internal test set (95% with high family score of which 100% were concordant), external set (75% with high family score of which 99% were concordant), Discovery-Distant (64% with high family score of which 92% were concordant or methylation-based reclassification was favored) and Discovery-Unclassified (37% with high family score, of which 44% had orthogonal based on which classification was favored). This is likely a consequence of these sets containing an increasingly greater number of cases with methylation profiles that were not present in the training set (most prominently in Discovery-Unclassified) or contained cases which were excluded from the well-defined reference clusters because of their non-proximity to the UMAP clusters (Discovery-Distant). This was found to be consistent with the CNS and sarcoma classifiers, the performance of which degrades when previously not-trained neoplasm types and neoplasms with high contamination by non-neoplastic tissues are tested.

This study has several limitations. Due to their small number, many renal neoplasm types that are genomically-defined such as ones with alterations in ALK, TCEB1, TFE3, TFEB, SDH, and others, were excluded. Some neoplasms, such as oncocytoma and LGOT, were represented by a very small number of cases. Confirmation of the validity of the methylation classification in instances with a discrepancy between the initial diagnosis and classification by the trained classifier was challenging because H&Es, comprehensive histologic review, and molecular analysis were not available for many cases, while immunohistochemistry was almost never available. A consequence of this was the inability to evaluate these discrepancies, including, for example, the “reclassification” of an initial diagnosis of AML case to a ccRCC methylation classification (case 2217 in Chopra validation set). Finally, the decision to include specific epithelial entities in the clear cell, papillary, and oncocytic group entities is convenient but arbitrary. Future iterations of a kidney methylation classifier may need an extensive and fully characterized set of reference and validation cases.

DNA methylation is a viable marker for the identification of new renal cancer types and a realistic medium for the development of a prospective cancer classification. Such tool can become part of a clinical workflow. Maintenance of such a tool will require interinstitutional collaboration, and continued addition of increasingly rare entities.

### Example 2: DNA Methylation-Based Classifier for CNS Tumors

### Development of a DNA Methylation-Based Classifier

The development of a DNA methylation-based classifier for CNS tumors commences with the meticulous collection of tumor samples from patients. These samples underwent a rigorous process involving DNA extraction and DNA methylation profiling, carried out using Illumina Infinium methylation EPIC technology. The resultant data underwent comprehensive preprocessing, specifically designed to eliminate noise and batch effects. This meticulous preprocessing is paramount in ensuring the reliability and integrity of all subsequent analyses. To construct the CNS classifier, a reference set consisting of 7,467 samples was initially compiled. This compilation was achieved through an extensive prescreening process, which was further refined through UMAP optimization.

### Feature Selection and Model Training

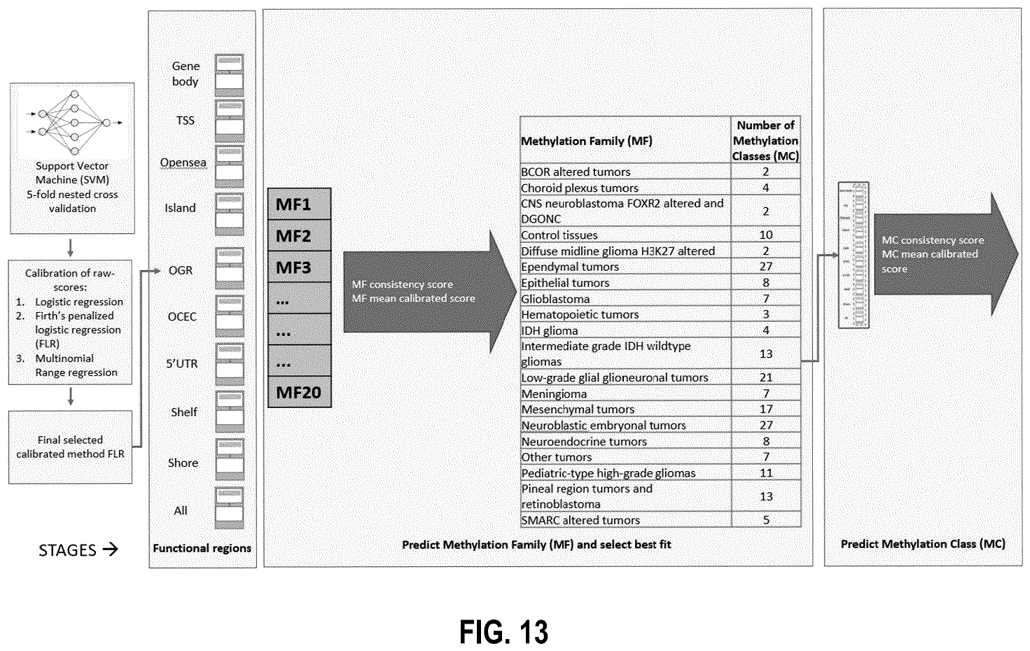

Subsequently, a stratified classification methodology was implemented, which entailed the development of multiple classifiers operating at both the family and class levels, as illustrated in FIG. 13. This stratified approach was instrumental in enhancing accuracy and in assessing the performance of distinct genomic regions concerning classification. In this context, a “family” refers to cohesive groups of classes that exhibit similar methylation profiles. These groups are identified through unsupervised clustering and UMAP optimization. The classification approach adopted follows a two-step process. Initially, an appropriate training algorithm and calibration model were carefully selected and were uniformly applied across all classifiers. Following this, a collection of multiple classifiers was trained, each utilizing distinct sets of the most variable 10,000 probes. These probes are derived from biologically significant genomic regions, including Gene body, Island, Opensea, Other genomic region, Open chromatin region, Shore, Shelf, TSS, 5′UTR, and the whole array probes.

### Model Evaluation and Validation

To ensure consistency, the support vector machine (SVM) algorithm was employed for training all classifiers, with each classifier undergoing a 5 by 5 nested cross-validation (CV). In the first step, all samples were randomly divided into five sets. For each outer CV loop, one of these sets was designated as the test set, while the remaining four sets were combined to form the training set. Subsequently, for each training set, all samples were further divided into five subsets. Out of these five inner sets, four were utilized for the training set, and one served as the test set. This entire process was iterated five times to generate the inner CV loops. Consequently, a total of five outer loops were established, each yielding five inner loops. The outer loops were employed to evaluate the model's generalization capabilities, while the inner loops played a pivotal role in preventing over-fitting during the calibration phase. Notably, the test sets of the inner loops were employed to construct the calibration model, a prudent approach that ensured the model's robustness and performance, rather than relying solely on either the inner or outer training sets. All models were rigorously tested and assessed for performance. Mean error (ME) was computed for each classifier, where ME is defined as the proportion of incorrectly classified cases across all classes divided by the total number of cases. Three distinct calibration methods were compared, consistent with previous research: (i) Platt scaling, executed through LR fitting, (ii) Firth's penalized likelihood LR (FLR), and (iii) ridge-penalized multinomial logistic regression (MR). The SVM model was configured with a linear kernel, and to enhance accuracy, all calibration models underwent testing. Ultimately, the SVM-MR model emerged as the optimal choice, exhibiting error rates ranging from 0 to 0.02 at the family level and 0 to 0.12 at the class level.

The subsequent critical phase of classifier development involved the training of multiple classifiers at both family and class levels, culminating in the derivation of the final output as the mean calibrated score for any given test sample. A total of 20 families and 198 classes were annotated from the entire reference set of 7,467 samples. At the family level, ten classifiers were trained utilizing the most variable probes from ten distinct functional regions on the array. Subsequently, we trained ten classifiers for each individual family to predict the appropriate class. In sum, we developed a total of 210 mini classifiers: 10 at the initial level for family classification, and an additional 200 (20 families times 10 classifiers per family) for class classification.

The performance of the DNA methylation-based classifier is assessed using validation datasets to ensure its accuracy, sensitivity, specificity, and robustness. Cross-validation and independent validation cohorts are essential to confirm the classifier's reliability.

### Clinical Applications

The primary clinical application of the DNA methylation-based classifier is the accurate classification of CNS tumors into molecular subtypes. This information can aid in treatment planning, as certain subtypes may respond better to specific therapies.

In some examples, the classifier may be integrated with other molecular data. Combining DNA methylation data with other molecular information, such as gene expression or somatic mutations, may lead to more comprehensive and accurate classifiers.

In other examples, the classifier may be used as part of personalized medicine. Tailoring treatment strategies based on the unique molecular profile of each patient's tumor holds great potential for improving therapeutic outcomes.

In further examples, the classifier may be used with large-scale data sharing: collaborative efforts to collect and share DNA methylation data from diverse CNS tumor cohorts can enhance the development and validation of classifiers.

The development of a DNA methylation-based classifier for CNS tumors represents a significant advancement in the field of neuro-oncology. This approach offers the potential for more accurate tumor classification, better treatment stratification, and improved patient outcomes.

Complementary Classification Approach to Resolve Low-Score Results from the DKFZ (Heidelberg) Methylation Classifier for CNS Tumor Diagnostics

As described above, a complementary classification approach (“Bethesda classifier”) was developed with a new reference set of several thousand samples, utilizing the extent of ˜850 k probe set on the EPIC array. In this approach, multiple (10) classifiers were created by dividing the probes into 10 independent bins, allowing dual measurements of both an overall confidence score, as well as a consistency score of each bin prediction.

A multiple bin-based consistency score (CS) was established for each class. In an alternate approach non-negative matrix factorization (NMF) was found to be highly useful to find new tumor entities.

The EPIC array was divided into 10 bins based on functional genomic locations. The different functional regions can be selected from the group consisting of Gene body, Island, Opensea, Other genomic region, Open chromatin region, Shore, Shelf, TSS, 5′UTR, and whole array probes. SCMER (single cell manifold preserving feature selection) was used for feature selection and a neural network was used to train classifiers. Each bin works as an independent classifier.

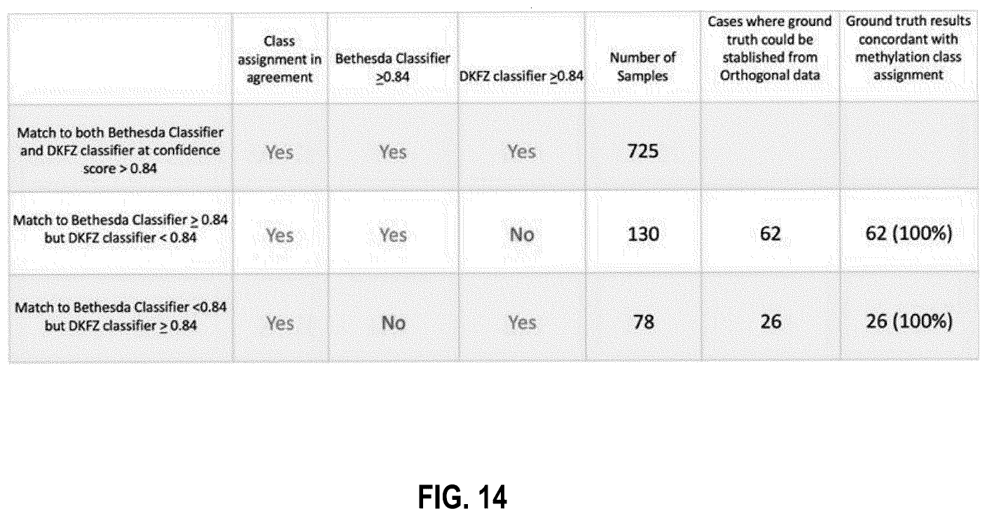

The Bethesda classifier was compared to the classifier developed by the German Cancer Research Center (DKFZ). FIG. 14 shows a comparison of cases which received >0.84 confidence score in at least one classifier. 725 cases received high score in both. Of the remaining, 130 received high score in our classifier. Of these 130 cases, 62 classes could be confirmed and verified using orthogonal data (tumor type-specific mutation, fusions, and/or copy number profile. Conversely, 78 cases received high scores in the DKFZ classifier but not Bethesda classifier, of which 26 could be verified. Results indicate that each classifier can complement each other to gain confidence in prediction when scores of either classifier are low.

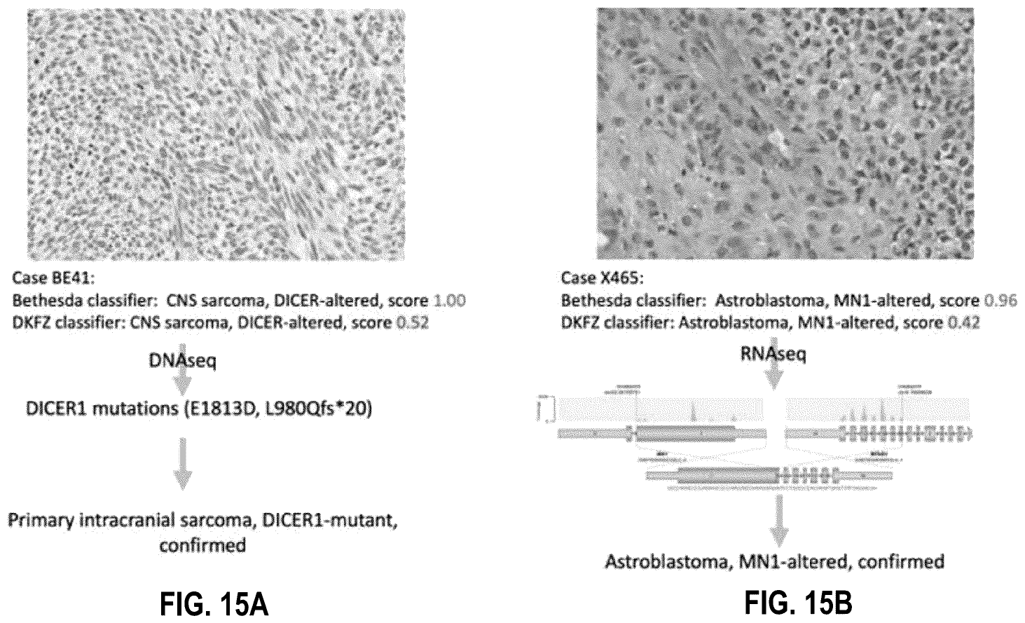

FIG. 15A and FIG. 15B show two example cases where the Bethesda classifier output is a high score and the DKFZ classifier output is a low score, and the results were consistent with ground truth confirmed by genomic data.

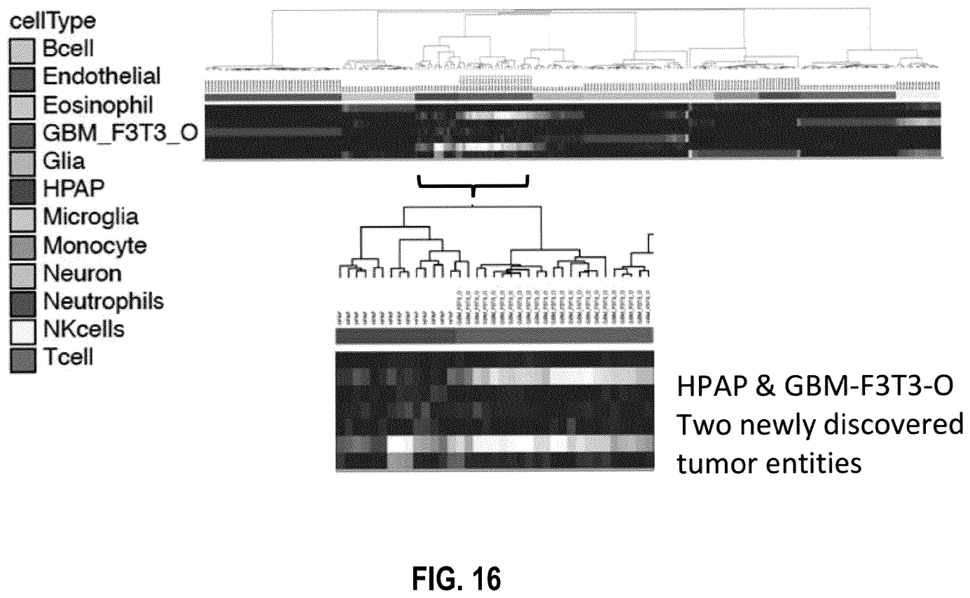

FIG. 16 shows non-negative matrix factorization as a novel approach to discover new tumor entities. For example, HPAP and GBM-F3T3-O were two newly discovered tumor entities.

Low scores are common in methylation profiling. The use of multiple classifiers may provide additional opportunities to gain confidence in low-score cases from the DKFZ classifier. The complementary classifier that was developed utilizes much of the EPIC array. 130 cases were identified with highly confident scores on Bethesda classifier, but <0.84 on DKFZ classifier. ‘Ground truth’ was assessed via orthogonal data on 62 of these cases and confirmed that methylation results were accurate. Use of a complementary classifier may resolve some additional cases with low scores on DKFZ classifier. Therefore, non-negative matrix factorization may be a novel approach to explore low scored samples with consistent methylome patterns.

### Example 3: DNA Methylation-Based Classification of Hematolymphoid Neoplasms

### Sample Selection

Samples included excisions, core biopsies, bone marrow, peripheral blood. A reference set of samples were generated. An exploration set was composed of 310 samples that were available at time of reference set generation but were not used in the reference set. In addition, an external validation set was composed of 173 additional samples, which were completely independent from reference set generation and classifier development. All cases in the reference set had undergone rigorous review by expert hematopathologists and tumor-type specific molecular testing for the identification of the relevant alterations, whenever possible. For each specimen in the reference set, a maximal tumor cell content was aimed for. Ethics approval for the work was obtained in the form of IRB-approved protocols to ESJ and KA.

### Methylation Array Processing and Quality Control

Genome-wide DNA methylation profiling was performed with the Infinium Methylation EPIC BeadChip array (Illumina, CA, USA). Extracted DNA was analyzed following the manufacturer's protocol. Additional profiles were obtained from publicly available datasets. IDAT files were processed using the R (version 4.1.0) programming language. The bioinformatics UMAP workflow was built using ‘meffil’ package, providing data loading, quality control (QC), normalization, and probe filtering. Various QC metrics including signal intensities, control probe means, detection p-values, and low bead counts, were extracted from the QC summary. Samples with missing information or those flagged as failing default QC metrics were identified and removed from consideration.

Principal Component Analysis (PCA) was used for the QC objects generated by “meffil.plot.pc.fit” function to estimate the best number of principal components to adjust normalization parameter in quantile normalization, which is performed on the QC objects using the “meffil.normalize.quantiles” function. Beta values were extracted from the normalized objects using the “meffil.normalize.samples” function. Dimensionality reduction was performed with the top 15000 probes with the highest standard deviations.

### Class Visualization and Classifier Development

To develop the classifier, a reference set (n=1156) was created based on prescreening and UMAP optimization. After the dataset was finalized a stratified classification approach was adopted which includes development of multiple classifiers at the family level and class level. This strategy included dividing groups identified on UMAP into one of seven ‘families’ based on overall similarity in methylation signatures. Once the families were defined, 7 distinct family-level classifiers were created, with a goal to improve resolution among classes within a family where methylation profiles were similar. Survival analysis was performed using Kaplan-Meier analyses to identify patient survival differences as they relate to specific methylation-defined classes.

UMAP code utilized the “meffil” package and supporting libraries for quality control, normalization, and probe filtering of DNA methylation data. The code followed a stepwise approach, starting from data loading and preprocessing, performing QC analysis and filtering, and finally generating normalized beta values.

The classification approach consisted of two major steps. In the first step the appropriate training algorithm and calibration model were selected to apply to all classifiers uniformly. In the second step, a collection of multiple classifiers was trained on a different set of most variable 10 k probes from biologically important array regions such as Gene body, Island, Opensea, Other genomic region, Open chromatin region, Shore, Shelf, TSS, 5′UTR and whole array probes. A support vector machine (SVM) algorithm was implemented for all the classifiers in the training series, and each classifier was trained with a 5 by 5 nested cross-validation (CV).

In the first step, all samples were randomly divided into 5 sets of samples and for each of the outer CV loops, one of these sets was used as the test set and the other four were used as the training set. Next, for each iteration, all samples were further subdivided into 5 subsets, and the whole process was repeated 5 times to generate inner CV loops. Hence, total 5 outer loops were made with each derived 5 inner ones. The outer loops were helpful to evaluate the generalization of the model, while the inner loops were informative to minimize over-fitting during the calibration step. The test sets of the inner loops were used to develop a calibration model. All models were tested performance evaluation.

### Classifier Calibration

Three different calibration methods were compared to get calibrated scores as described previously, (i) Platt scaling, implemented by fitting logistic regression (LR) (ii) Firth's penalized likelihood LR (FLR) and (iii) ridge-penalized multinomial logistic regression (MR). SVM with a linear kernel was used. To improve its accuracy, all calibration models were tested and SVM-FLR model was selected as the best model with a range of errors between 0.003 to 0.005 at the family level and 0 to 0.12 at the class level. The second major step of classifier development was to train multiple classifiers at both family and class levels and get the final output as a mean calibrated score for any test sample. A total of 7 families (MCF1-MCF7) and 44 classes were annotated from the whole reference set (n=1156). At the family level, 10 classifiers were trained using the most variable probes from ten different functional regions on the array. In the next step, we trained 10 classifiers for each individual family to predict the appropriate class. Overall, we trained 80 mini classifiers (10 at the first level to classify the family and 10×7=70 for each individual family to classify the class). Thresholds were calculated for calibrated scores using a Youden index on cross-validation output. The threshold for the family-level classifier was >0.96 and for the class-level classifier was >0.89. Finally, the accuracy of all classifiers was validated in independent samples not included in the reference set for training the classifier (n=483).

### Tumor Purity, and Copy Number Determination

The estimated tumor purity for all reference cases was computed using the R package RF_purify. For the illustrations, the predictions obtained with the method ‘RF_purify_estimate’ were used. Copy number alterations of genomic segments were inferred from the methylation array data based on the R-package conumee after additional baseline correction (https://github.com/dstichel/conumee). Summary copy number profiles were created by summarizing these data in the respective set of reference cases for each methylation class.

### Hematolymphoid (HL) Neoplasm Reference Set

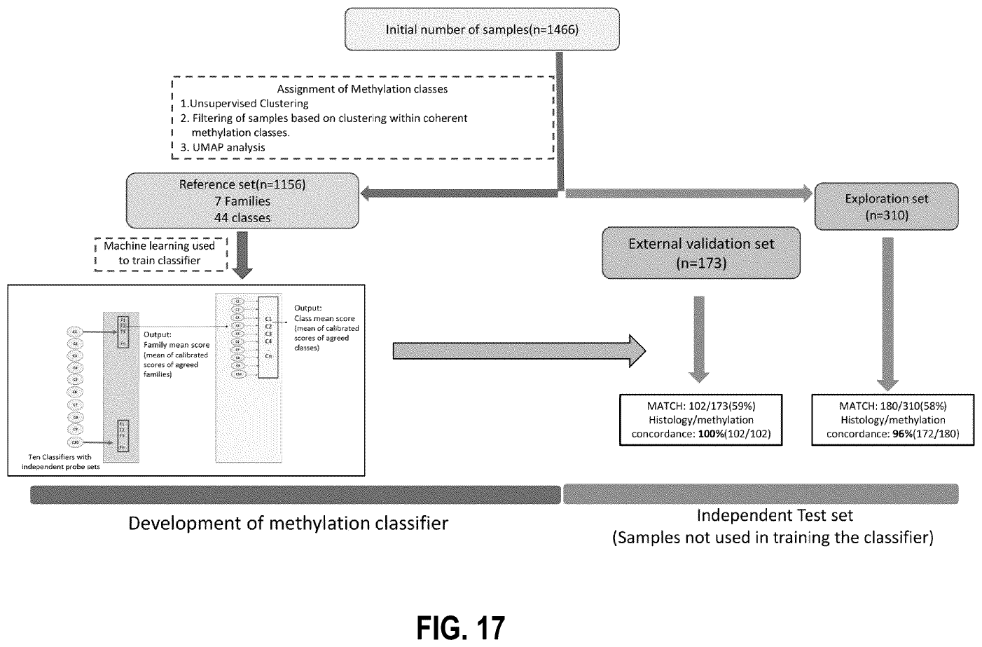

From the initial training data, we selected 1156 samples, representing well-defined methylation classes for our reference set. Remaining 310 samples formed the exploration set and an additional 173 samples comprised the independent validation set (FIG. 17).

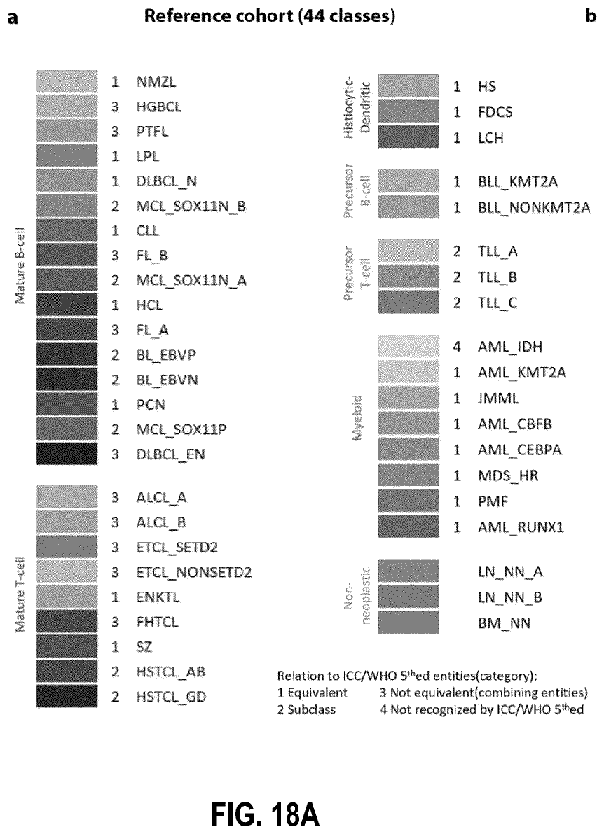

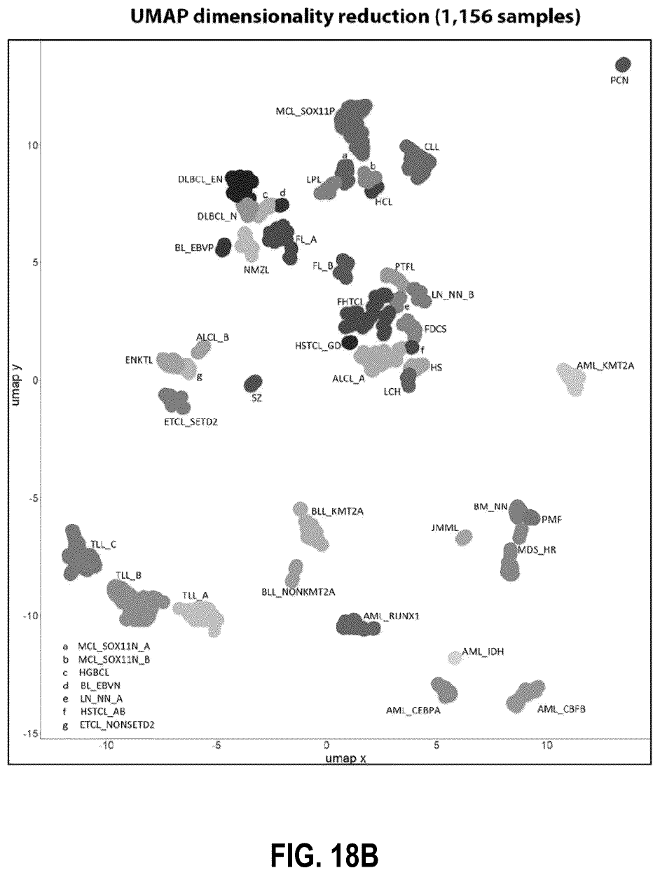

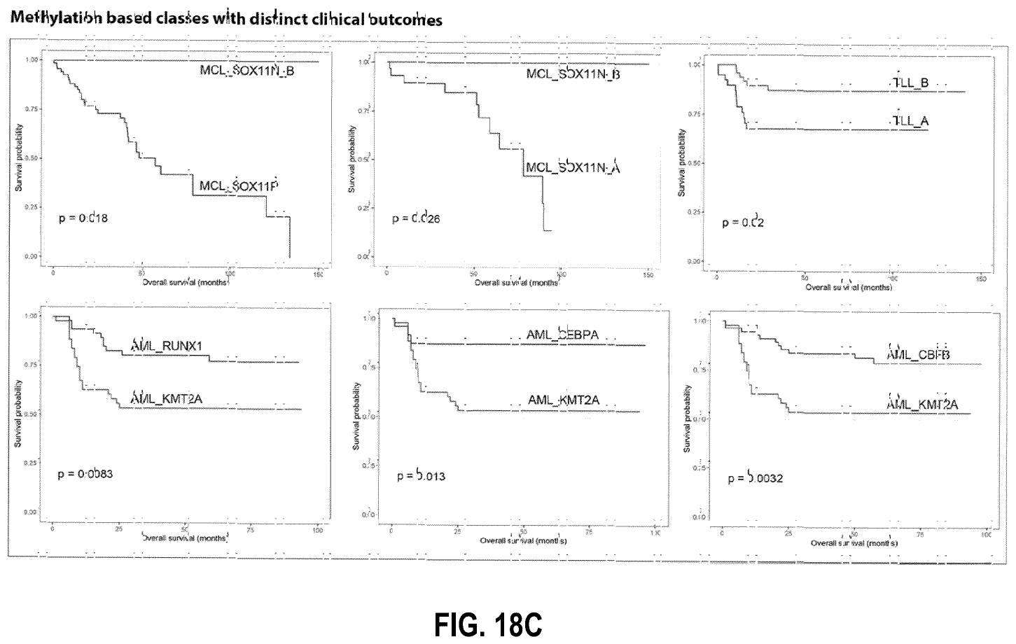

Classes defined in the reference set included 43 HL entities recognized by the WHO 5th edition and/or ICC. The methylation classes that emerged represented either (i) a one-to-one correspondence of class to tumor type; (ii) distinct DNA methylation classes within one histopathological tumor type (i.e., tumor subtypes); or (iii) a methylation class comprising multiple histopathological tumor types. This led to the designation of 41 tumor types characterized by distinct methylation profiles (FIGS. 18A and 18B). Twenty of these were associated in a one-to-one manner to a WHO 5th edition/ICC entity (category 1). Ten methylation classes represented subgroups within an ICC/WHO 5th edition entity (category 2). For 10 tumor classes, multiple histopathological tumor types were collapsed into a single methylation class, where methylation signatures were highly similar (category 3). Finally, one class (AML_IDH), a methylation class (category 4) was found that is not yet defined by current WHO or ICC classifications (FIG. 18A). We additionally included non-neoplastic conditions, including reactive lymph nodes and non-neoplastic bone marrow, resulting in a total of 44 methylation classes.

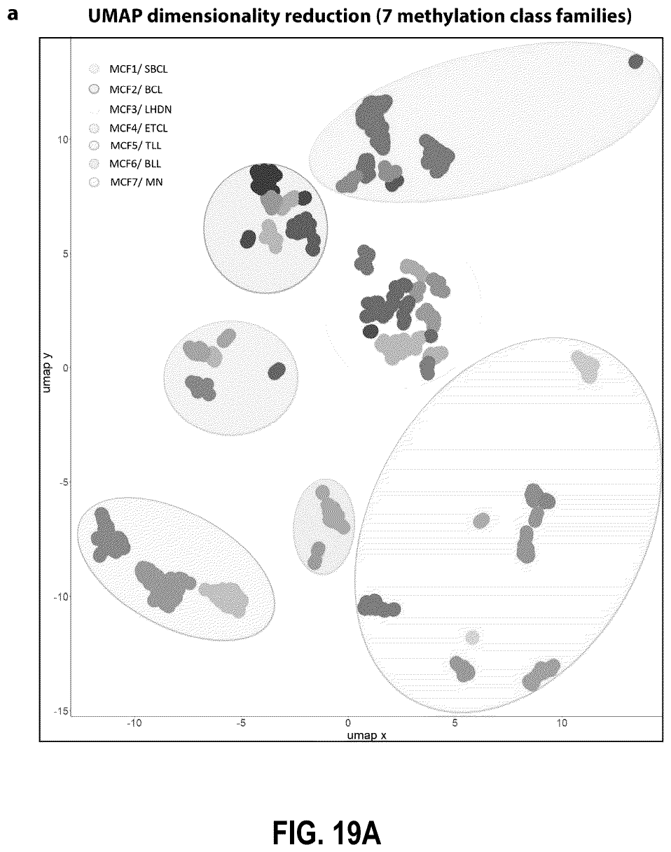

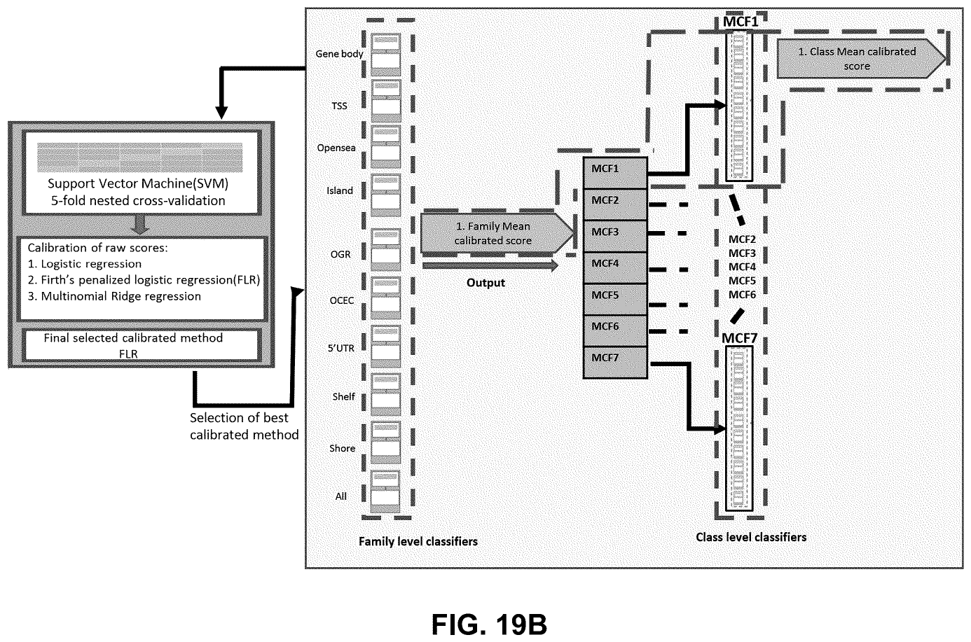

The stability of methylation classes was analyzed by iterative random down-sampling of the reference cohort and indicated high stability of the groups. Testing for confounding batch effects within the reference cohort did not reveal unexpected confounding factors. Patient survival times were compared where classes defined subtypes of recognized tumor entities and found that a subgroup of SOX11-negative mantle cell lymphoma (MCL_SOX11N_B) showed improved patient outcomes when compared to the other SOX11-negative methylation subgroup (MCL_SOX11N_A). In addition, the MCL_SOX11N_B subgroup showed improved outcomes when compared with as well with SOX11-positive MCL (MCL_SOX11P). As additional examples, the 2 methylation subclasses of T-cell lymphoblastic lymphoma that emerged from the data (TLL_A and _B) showed a significant survival difference, with TLL_A showing a worse prognosis and a lower proportion of TLX1 alteration (0/24 tested cases) compared to TLL_B, with improved survival and a higher proportion of TLX1 alteration (17/41 tested cases), in line with prior studies. Prognostic relationships were also found in methylation-defined subclasses of AML (FIG. 19C), where the KMT2A rearrangement-enriched cases showed a worse prognosis, when compared to AML with alterations in CEPBA, RUNX1, CBFP, overall demonstrating the existence of methylation signatures that correlate with clinically relevant genomic alterations.

### Classifier Development

Application in routine diagnostics requires a measure of confidence for a specific match, in addition to being fast and reproducible. To achieve this, the support vector machine model algorithm was employed to build a stratified classifier, where classes in the reference set were divided into 7 families (FIG. 19A). Each family represents a group of classes with similar methylation profiles identified by unsupervised analyses. A stratified classification approach was adopted, which includes development of multiple classifiers at the family level and class level (FIG. 19B), to promote accuracy. The overall design classified samples at first on family level followed by a family-specific classifier, with every sample receiving a calibrated score for both family and class. (FIG. 19B).

Cross validation, of all the classifiers showed overall low error rates. However, an increased error rate was noted for the MCF2/SBCL family as compared to all other individual family classifiers (MCF1-MCF7). Thresholds were calculated for calibrated scores using a Youden index on cross-validation output. The threshold for the family-level classifier was >=0.94 and for class-level classifier was >=0.86.

### Validation of the Classifier

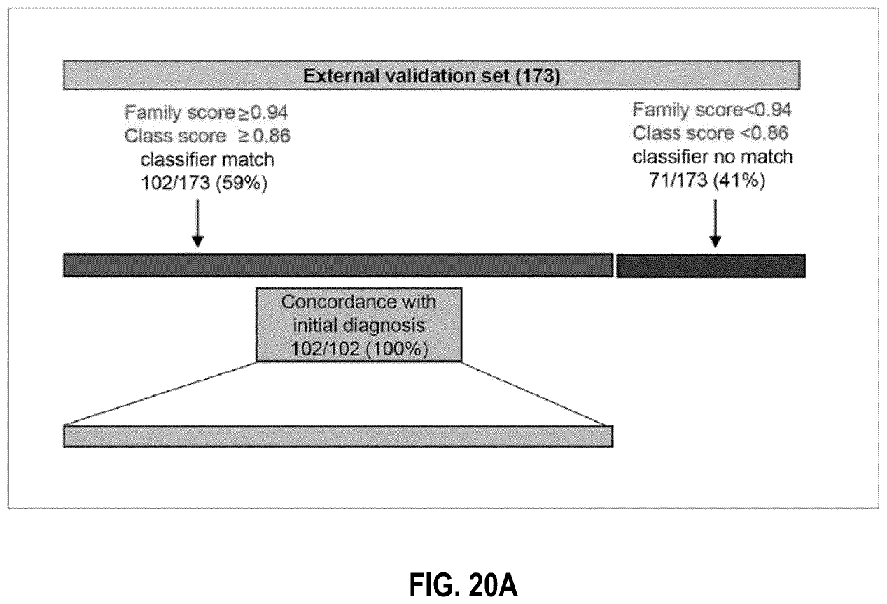

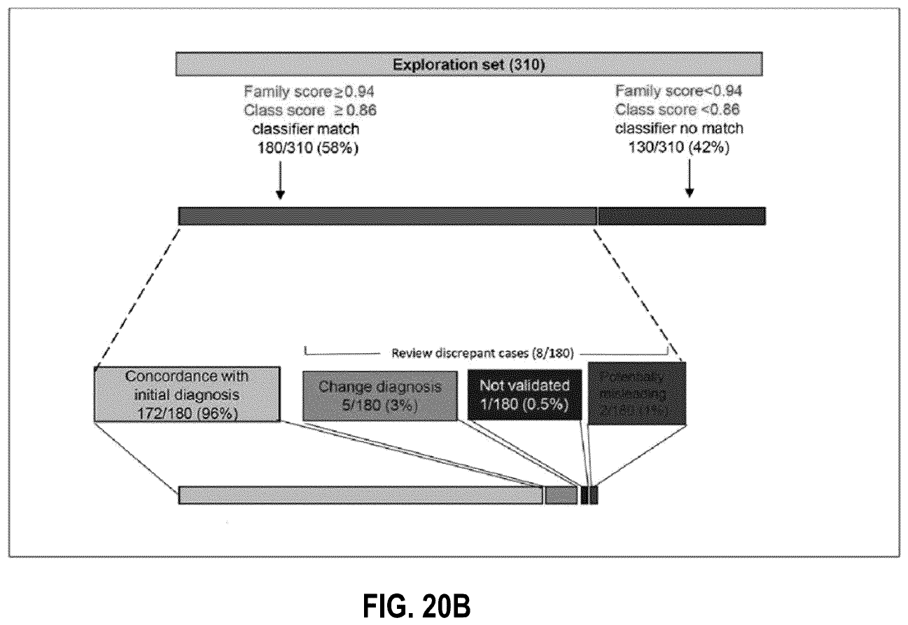

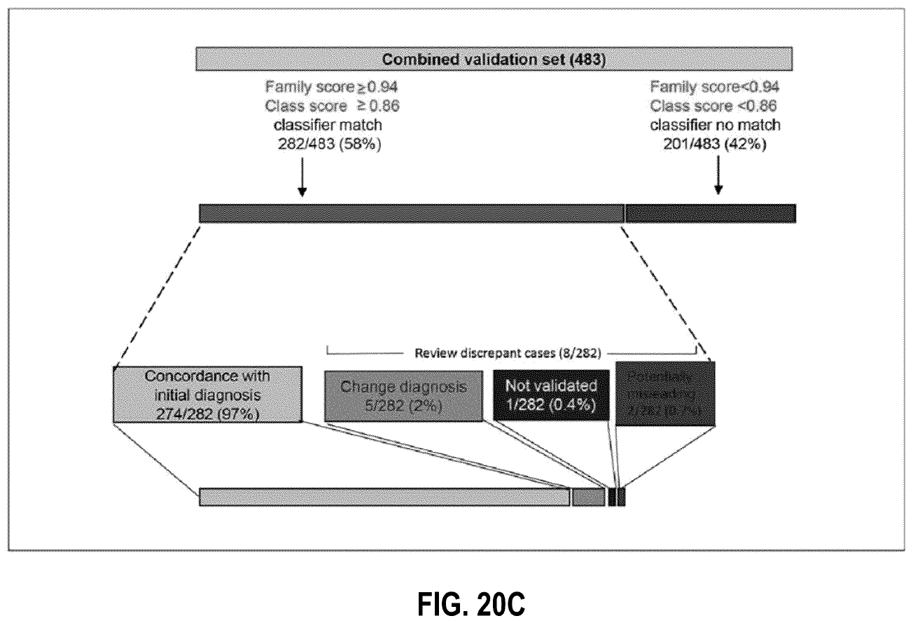

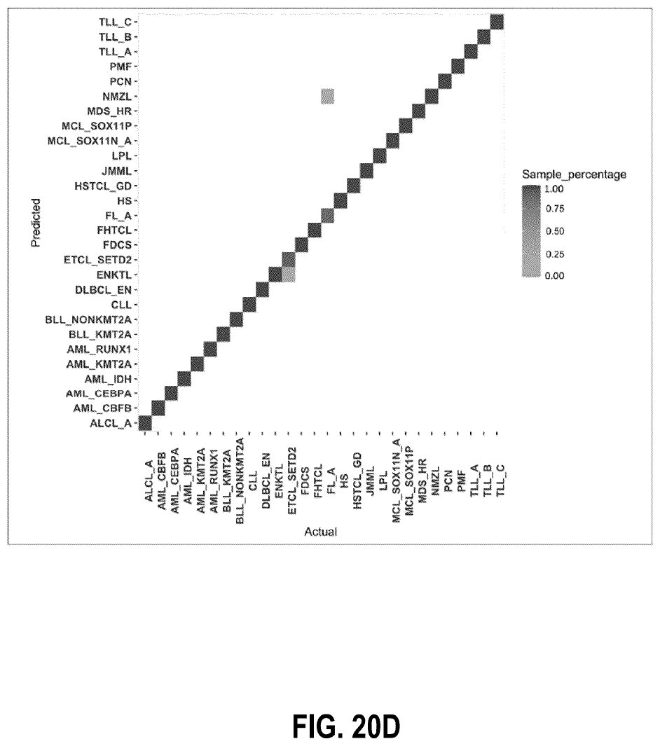

For evaluation of clinical utility, the classifier performance was validated in a test set (n=483) composed of samples that were not included in the reference set for training the classifier. Of these, 310 represented an exploration set (FIG. 20A), comprising samples that were pruned out in the initial evaluation because they did not group tightly with other samples on UMAP. An additional set of 173 samples, completely unseen in the reference set generation and classifier development, represented the external validation set (FIG. 20B). The cases, enriched for difficult-to-diagnose cases of lymphoid and histiocytic neoplasms received for referral were assigned as either ‘classifier match’ or as ‘classifier no match’ based on the established thresholds.

From the external validation set (FIG. 20A), a 100% concordance (102/102) was found with the institutional/pre-methylation diagnosis on the 59% (102/173) cases that were a classifier match with a classifier prediction family score>0.94 and class score>0.86. From the exploration set, (FIG. 20B), the classifier matched 58% (180/310) samples with a high concordance of 96% with the pre-methylation diagnosis with a classifier prediction family score>0.94 and class score>0.86. In total, 58% of profiled samples (282/483) matched to an established DNA methylation class. Ninety-seven percent (274/282) of these classifier-matched cases were concordant with the institutional (pre-methylation) diagnosis and 3% (8/282) were discrepant, which prompted re-evaluation of the diagnosis. (FIG. 20C).

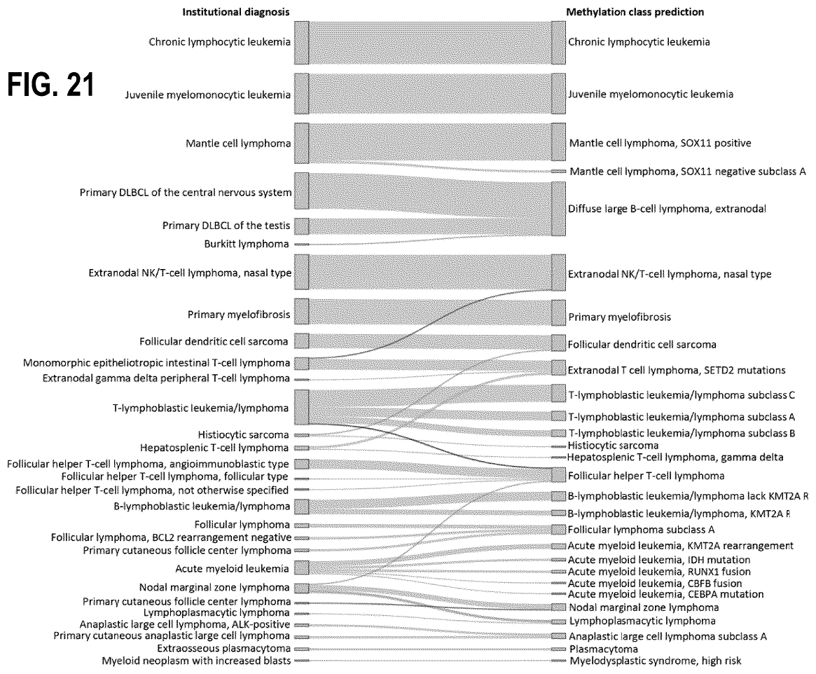

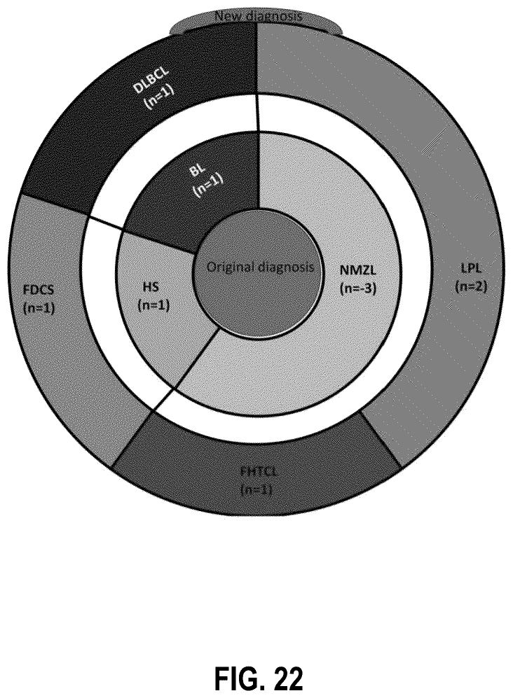

The discrepant cases were reviewed in detail, including methylation profile, the clinical chart, histology, IHC, copy number profile and/or molecular data, wherever possible, and identified as one of several categories: (i): discrepant-change in diagnosis; (ii) discrepant-potentially misleading profile or (iii) discrepant-not validated. (FIGS. 20D and 21). In 8 cases the classifier prediction differed from institutional diagnosis, with 5 cases diagnosis was changed in favor of the methylation class prediction (discrepant-change diagnosis; blue bars) (2% of the classifier match cases), one case where re-evaluation of diagnosis was not possible (discrepant-not validated; light blue bars), and two cases where the original diagnosis was retained, with a potentially misleading methylation profile (discrepant—potentially misleading; red bar). Establishing a new diagnosis (FIG. 22) was deemed to have clinical impact: Two cases with a pre-methylation diagnosis of nodal marginal zone lymphoma, matched to lymphoplasmacytic lymphoma (LPL), which prompted MYD88 testing. Both cases were positive for a MYD88 L265P mutation using droplet digital PCR (ddPCR) supporting and LPL diagnosis. In addition, copy number analysis showed a 6q deletion which is reported in 40-50% LPL tumors. An additional case with a pre-methylation diagnosis of nodal marginal zone lymphoma matched to follicular helper T-cell lymphoma (FHTCL), prompting additional review. Re-review was consistent with FHTCL, and the flat copy number profile that was observed in this case was consistent with this diagnosis. A case with a pre-methylation diagnosis of histiocytic sarcoma matched on the classifier to follicular dendritic cell sarcoma (FDCS), which prompted testing for CD21 and clusterin expression, both found to be positive on IHC. Copy number alterations on this case showed genomic complexity predominantly represented by alterations in multiple chromosomes including gain of chromosome 12 and 15 and multiple losses involving chromosome 9, 10,11,13,14 and 15 consistent with FDCS49. A breast mass biopsy with an original diagnosis of Burkitt lymphoma matched to diffuse large B-cell lymphoma, extranodal (DLBCL_EN), which prompted additional testing that showed a MUM1-positive/BCL-6-negative profile by IHC. Gains of chromosomes 7 and 13 were found, consistent with DLBCL, and together with the IHC profile, resulted in a change of diagnosis to DLBCL, NOS50. The initial diagnosis was retained against the predicted methylation class in 2 cases (<1% of the classifier match cases). The reasons for potentially misleading methylation class prediction in the latter cases, which passed the quality control steps, remains unknown, but in each case the overall findings were not deemed sufficient to change the diagnosis. One case where the methylation class suggested a different diagnosis relative to the pre-methylation diagnosis was considered ‘discrepant not validated’ as case could not be reviewed again due to unavailable tissue for additional testing.

### Tumor Purity and Classifier Performance

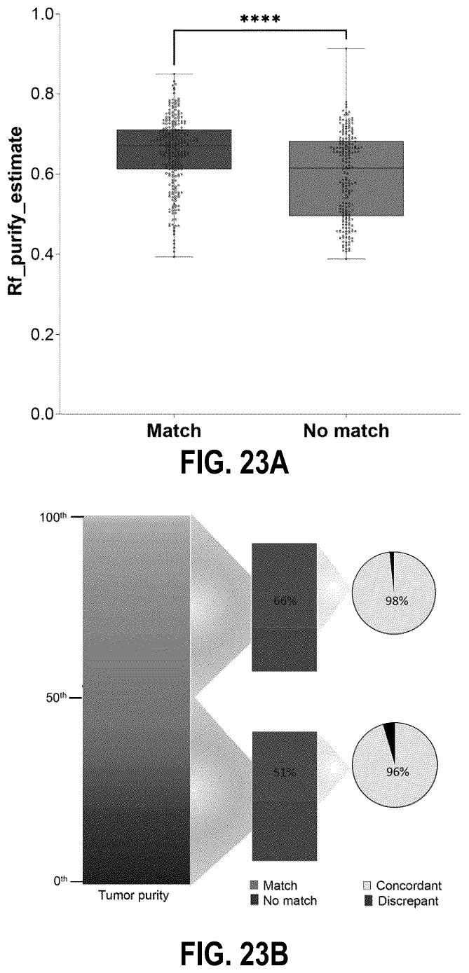

In all, 42% of tumors from the combined independent test set could not be assigned to a DNA methylation class above the calibrated thresholds (‘no-match’ cases), and a possible relationship of classifier match with tumor purity was examined. It was first attempted to establish the relevance of a previously described tumor purity metric (RF_purify_estimate) in hematolymphoid tumors. NCI cases profiled on the clinical service were accompanied by the hematopathologists' (ESJ and SP) estimate of tumor purity. No-match cases were found to have a significantly lower RF_purify_estimate scores compared to classifier-match cases (0.61 vs. 0.67, p value<0.0005, FIG. 23A). In addition, when the classifier results were stratified based on median tumor purity, 66% (159/241) high-purity cases were a classifier match, compared to 51% (122/241) of the low-purity cases that showed a classifier match (FIG. 23B). Overall, the results indicate that methylation classifier shows generally increased performance with increased tumor purity.

### Copy Number Profiling of Hematolymphoid Neoplasms

Independent from the methylation patterns used for classification, DNA methylation arrays allow for determining copy number alterations which are well described for some entities and are increasingly being studied for others and are being included in recent classifications of hematolymphoid neoplasms. Copy number variation (CNV) plots were generated from all hematolymphoid neoplasms of the reference cohort as described. Among the frequently observed copy number alterations for each methylation class, it was found that the poor-prognosis MCL SOX11N_A and MCL SOX11 P classes had more CNV alterations compared to the better-prognosis MCL SOX11 N_B subclass. Additional tumor classes showed CNV patterns in line with prior reports. While CNV alterations in hematolymphoid tumors are generally not pathognomonic on their own, when utilized in combination with methylation profiles, they can potentially add substantially to the diagnostic decision process, as has been shown for CNS tumors.

### Discussion

We here demonstrate that DNA methylation-based classification of HL neoplasms using a comprehensive machine learning approach could be a significant asset for clinical diagnosis and decision-making. A higher level of standardization has promise to reduce the substantial inter-observer variability observed in the diagnosis of HL neoplasms in current practice. Further, in contrast to traditional pathology, whereby all neoplasms may need to be assigned to a described entity even for atypical or challenging cases, the objective measure provided here allows for ‘no match’ to a defined class, which may signify that the tumor is difficult to place in a specific tumor entity. Conversely, tumors that are difficult to diagnose by conventional means may be resolved with the knowledge of the methylation match, as has been shown for CNS neoplasms. This information can also be of substantial value in highlighting that a neoplasm is not a typical example of a given differential diagnosis, and may rather belong to a rarer, yet to be defined class.

Employing DNA methylation-based categorization offers highly attractive features. DNA methylation is a robust technique and analyses can be performed on DNA extracted from FFPE tissues, allowing implementation in routine settings. This represents a clear advantage over RNA expression profiling, since gene expression values is often less stable due to sample age and tissue processing issues. Surgical excisions as well as core biopsies are amenable to DNA methylation profiling, as long as sufficient DNA (250ng) can be acquired. In addition, the detection of individual methylation patterns for HL entities is of special interest for the entities which lack a pathognomonic genetic alteration, which is true for approximately two-thirds of the entities of HL neoplasms currently recognized by the classifier. Further, the digital nature of methylation data facilitates easy exchange and will allow aggregation of extensive tumor libraries. This will likely result in the detection of exceptionally rare tumor classes that could only be discerned from collections of individual cases coming from multiple institutions. The inclusion of new classes could allow a prompt translation into diagnostic practice, almost certainly resulting in a more dynamic tumor classification.

While conceptually highly attractive, the current version of the classifier could not confidently assign 42% of the cases from the independent test set to a DNA methylation class. Several explanations may apply. First, the initial classifier does not include all possible HL tumor types. Several tumor types were not included due to rarity and insufficient samples to form a methylation class. However, development of this initial classifier provides a proof-of-principle. Second, similar to CNS tumors, accurate methylation-based classification in HL neoplasms depends in part, on tumor purity. In particular, some HL tumors contain high proportions of non-neoplastic inflammatory cells with resultant lower tumor purity. The sarcoma classifier reported similar findings with the inability to confidently match approximately 25% of validation set. However, the effect of tumor cell purity on classifier performance may be dependent on HL tumor subtype. One approach to overcome this problem may be to deconvolve methylation patterns typical for reactive lymphocytes, thereby accentuating patterns of the respective HL neoplastic entities. Also, some of the cases of the validation set were from public datasets and did not receive a centralized pathological reference review. While such centralized expert review would not affect the classifier performance, it likely would reduce the number of apparently discrepant cases. Lastly, limited sample numbers for some classes in the reference set may not fully represent the core methylation signatures of each class and therefore reduce the confidence scores for some cases. A future increase in the number of cases in the reference set, particularly for the rarer tumor entities, will likely enable the detection of more methylation subgroups, as was observed with the sarcoma classifier.

In summary, the DNA methylation profiling of hematolymphoid neoplasms identifies methylation classes that correspond to known clinicopathologic entities, including tumor subgroups with clinical and biological relevance. The disclosed classifier for methylation-based classification of hematolymphoid neoplasms provides an effective framework for the discovery of new tumor subtypes. New biological insights are also likely to be gained based on the interrelationships of tumor classes, and by closer examination of how differential DNA methylation affects tumor biology. For example, cases designated as HGBCL likely correspond to B-cell neoplasms with dark zone biology, recently delineated by digital gene expression profiling. A uniform implementation of this classification algorithm has great promise for standardization of tumor diagnostics across centers and across clinical trials. A web-based platform for classifier output on newly profiled samples may be used to facilitate access and promote future, more comprehensive versions of the classifier, with improved recognition of rare entities.

In some examples, the classifier may have better resolution of aggressive B-cell neoplasms, with delineation biologically distinctive subtypes. In addition, the classifier may employ deconvolution strategies to elucidate the neoplastic signal within the background of non-neoplastic cells, with a goal of improved diagnostic accuracy for samples with low tumor cell content. Finally, the classifier may incorporate additional HL tumor types in future versions of the classifier to increase the potential utility of methylation-based classification as a potential tool for clinical diagnosis and discovery.

### Example 4: DNA Methylation Profiles Identify Classes that Correspond to Tumor Types and Subtypes of Hematolymphoid(HL) Neoplasms

A study was designed to use DNA methylation profiles to identify classes that correspond to tumor types and subtypes of HL neoplasms. The study design included providing a reference cohort, providing the reference cohort to the classifier, and providing clinical implementation based on the output from the classifier.

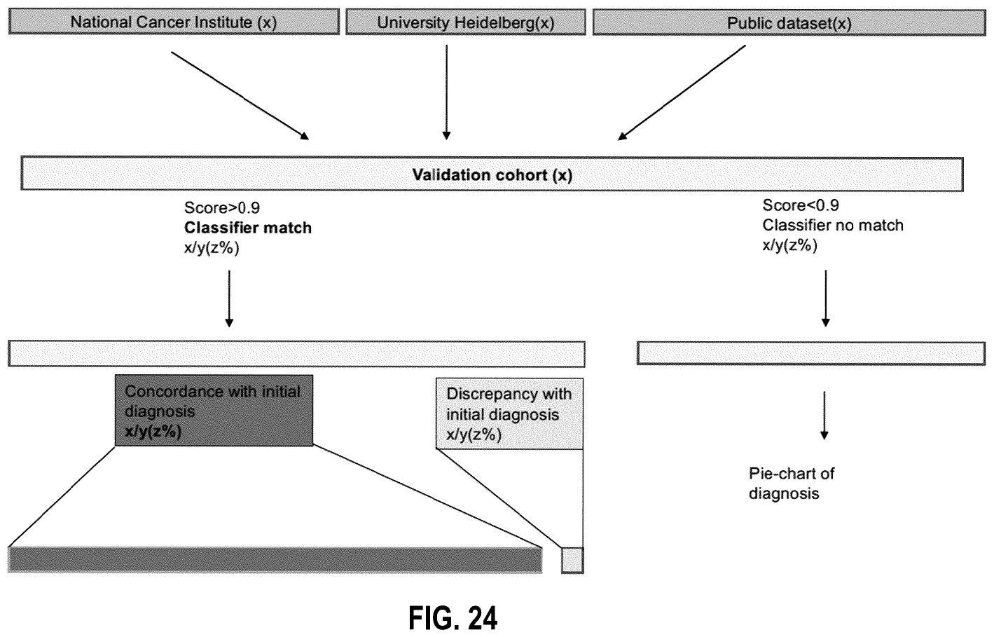

FIG. 24 shows an example flow chart of a clinical implementation of the study. For example, one or more datasets (e.g., National Cancer Institute, University Heidelberg, or other public datasets) may be provided as a validation cohort to the classifier. The samples with a confidence score >0.9 may be considered a classifier match. The samples with a confidence score <0.9 may not be considered a classifier match. Of the samples that are a classifier match, the classification from the classifier of some samples may be in concordance with the initial diagnosis while there may be a discrepancy between some classifications from the classifier and the initial diagnosis.

The establishment of the HL tumor reference cohort included 197 HL samples using an EPIC/850 k methylation array, 235 data sets from collaborators, and 832 public datasets for a total of 1281 DNA methylation profiles that were used to train a classifier using unsupervised clustering methods on Uniform Manifold Approximation and Projection (UMAP) dimensionality reduction.

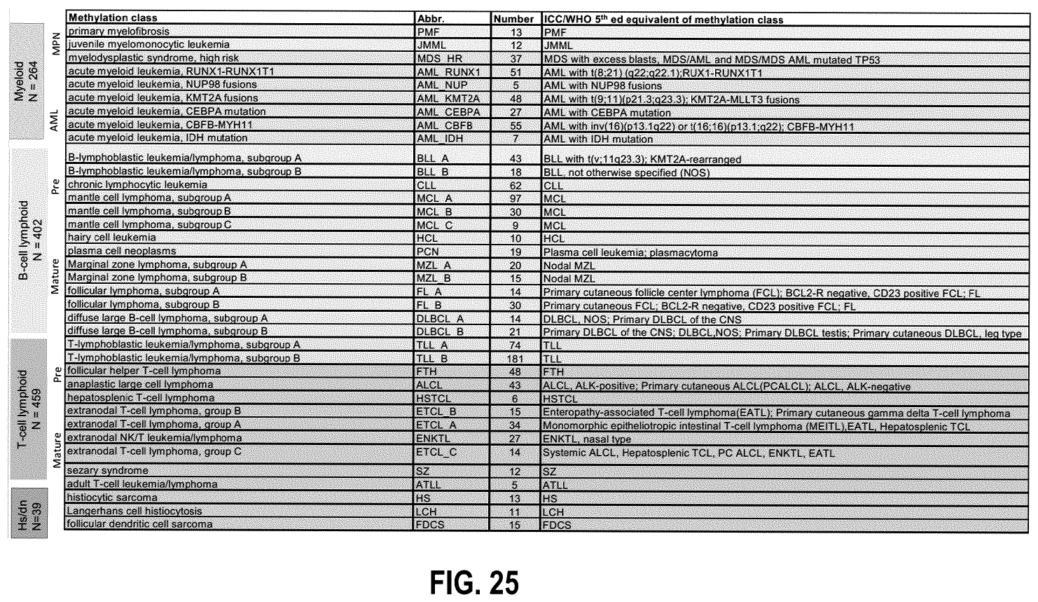

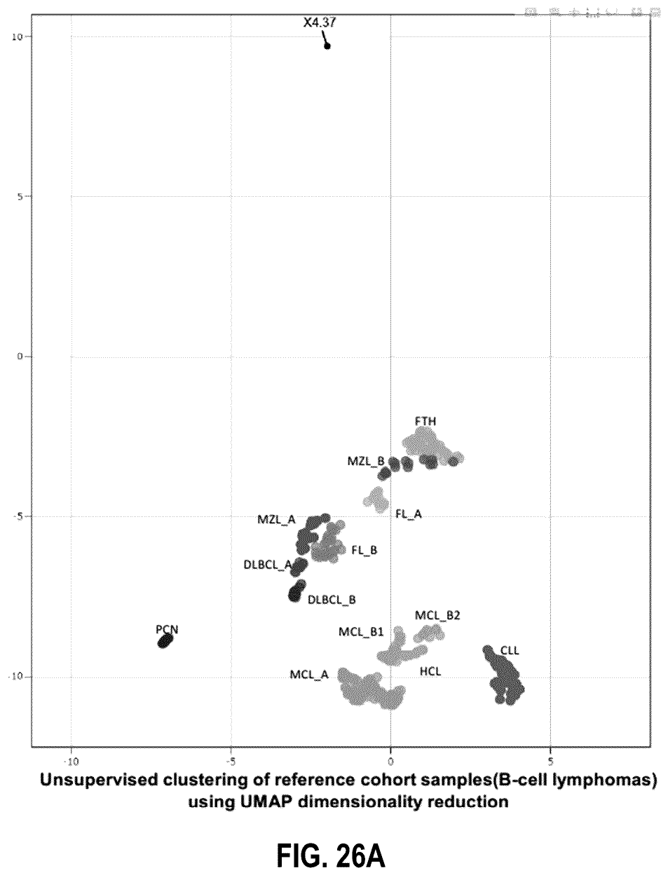

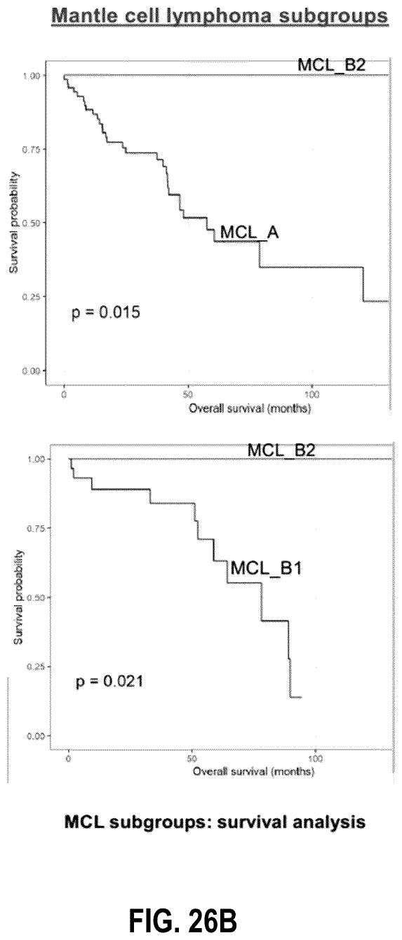

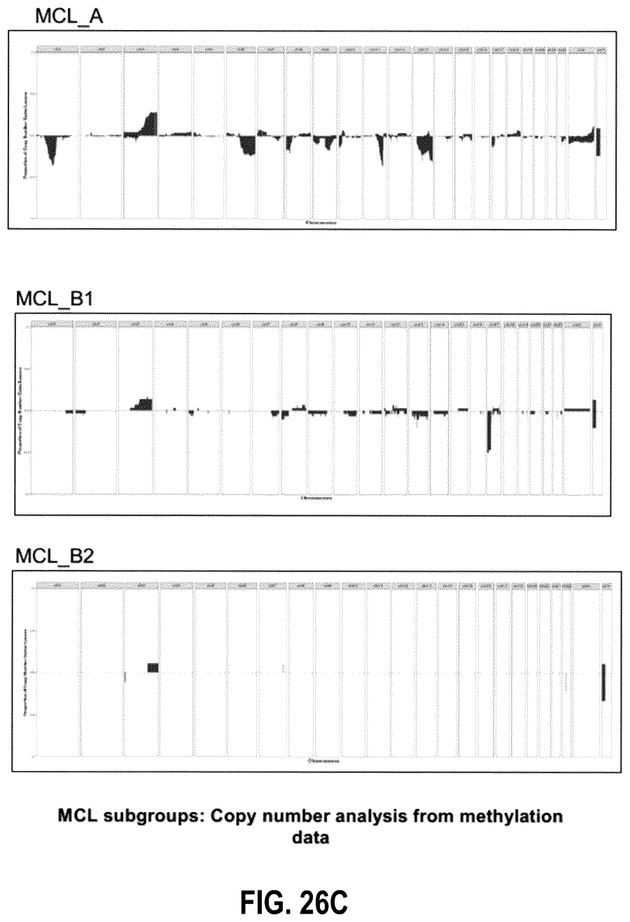

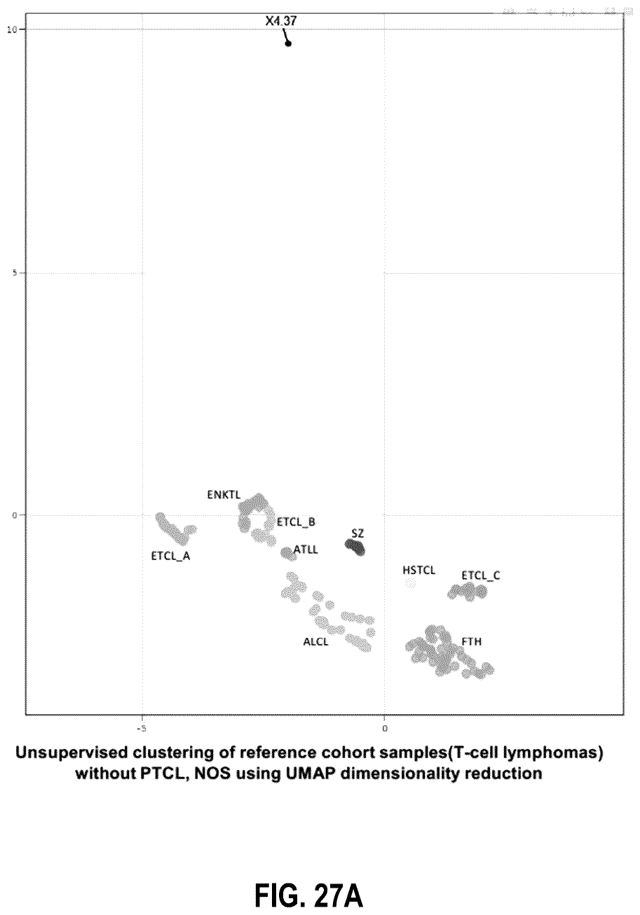

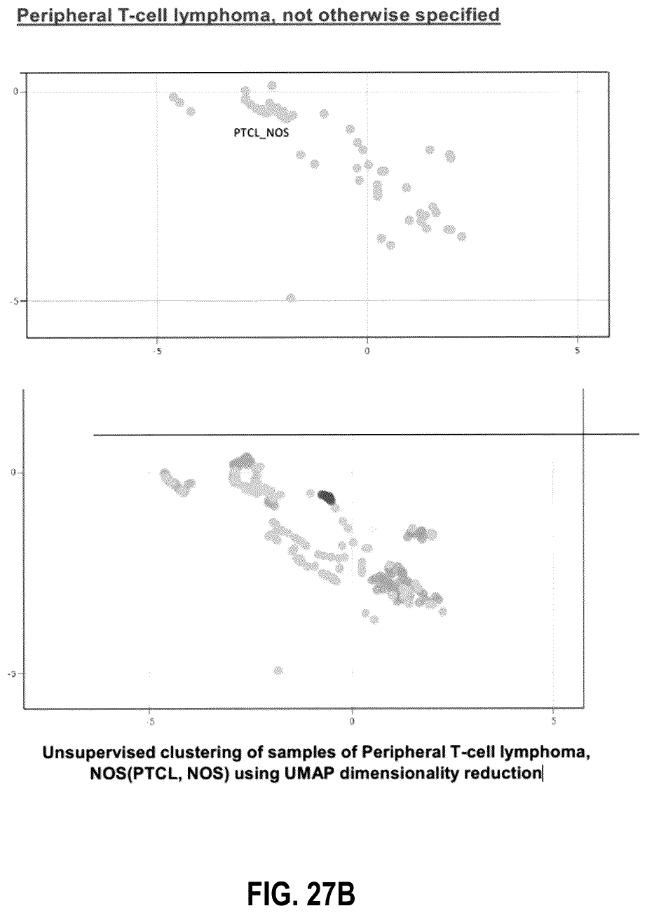

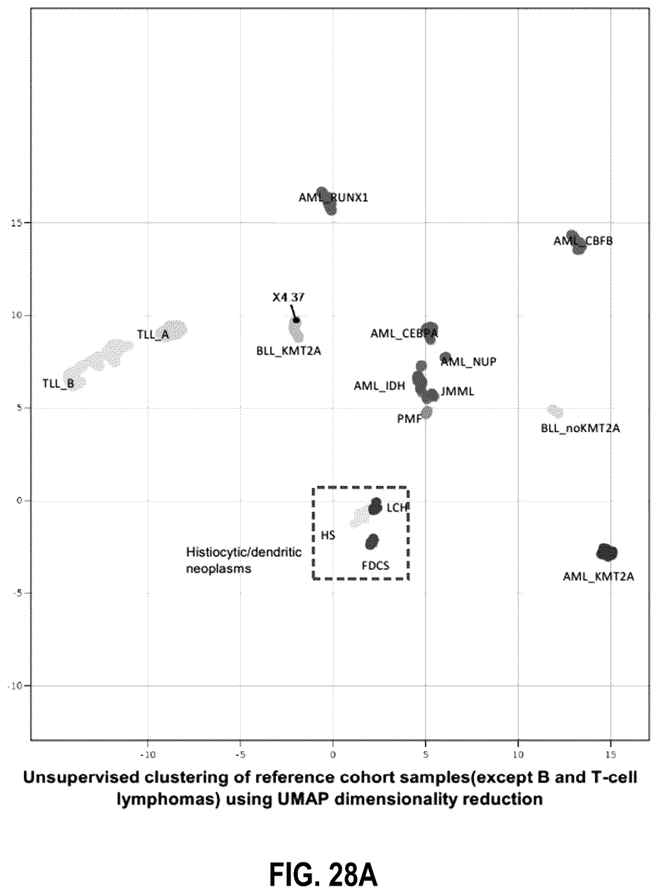

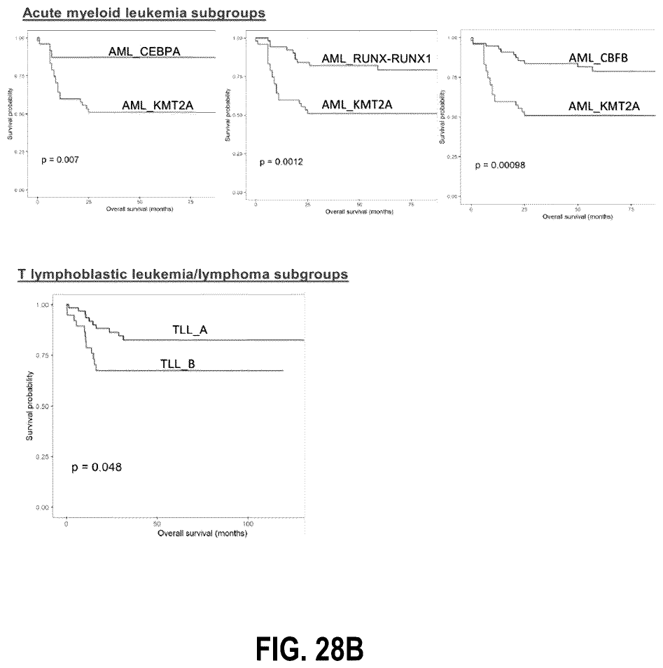

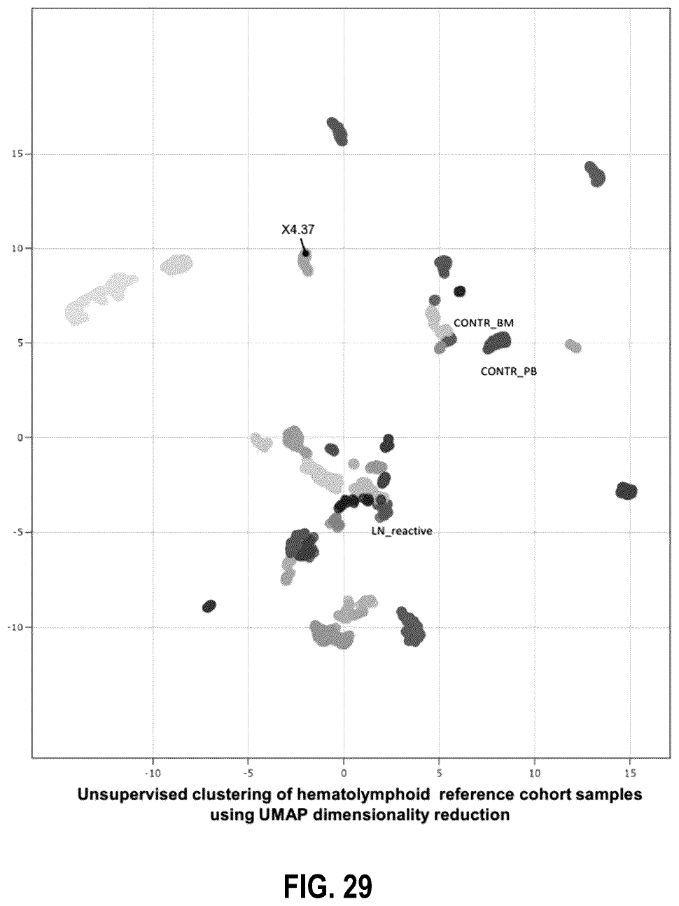

FIG. 25 provides a table of the methylation classes identified using the classifier. FIGS. 26A-26C shows an overview of classes of B-cell lymphomas, including the unsupervised clustering of the reference cohort samples using UMAP dimensionality reduction (FIG. 26A), survival analysis of MCL subgroups (FIG. 26B), and copy number analysis from the methylation data for MCL subgroups (FIG. 26C). FIGS. 27A-27B shows an overview of classes of T-cell lymphomas, including the unsupervised clustering of the reference cohort samples without PTCL, NOS using UMAP dimensionality reduction (FIG. 27A) and unsupervised clustering of samples of peripheral T-cell lymphoma, NOS (PTCL, NOS) using UMAP dimensionality reduction (FIG. 27B). FIGS. 28A-28B shows an overview of classes of other HL neoplasms, including the unsupervised clustering of the reference cohort samples (except B and T-cell lymphomas) using UMAP dimensionality reduction (FIG. 28A) and survival analysis of acute myeloid leukemia subgroups and T lymphoblastic leukemia/lymphoma subgroups (FIG. 28B). FIG. 29 shows the unsupervised clustering of hematolymphoid reference cohort samples using UMAP dimensionality reduction. The HL tumor reference cohort included 1281 samples and 39 methylation classes. In some examples, the methylation profiling using the classifier may be compared to a pathological diagnosis. In some instances, the comparison may result in a histological and/or molecular reevaluation.

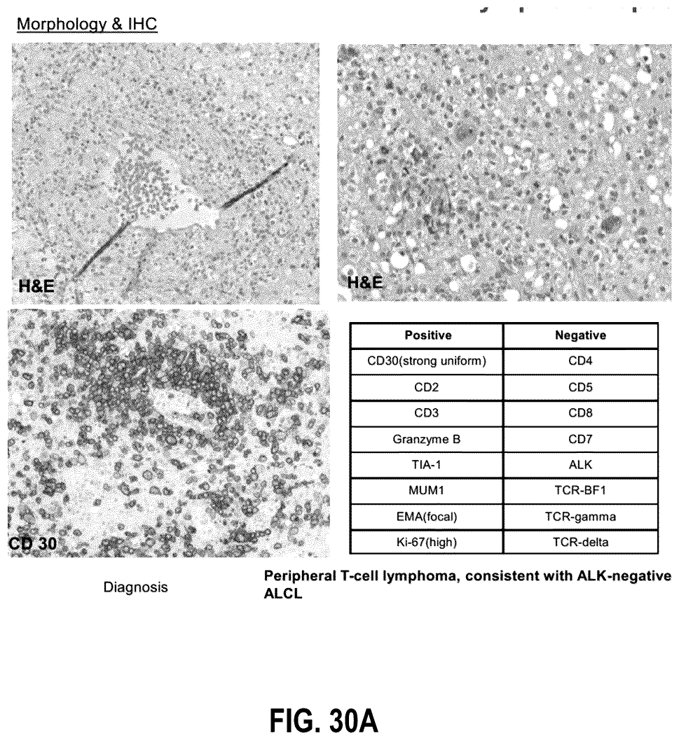

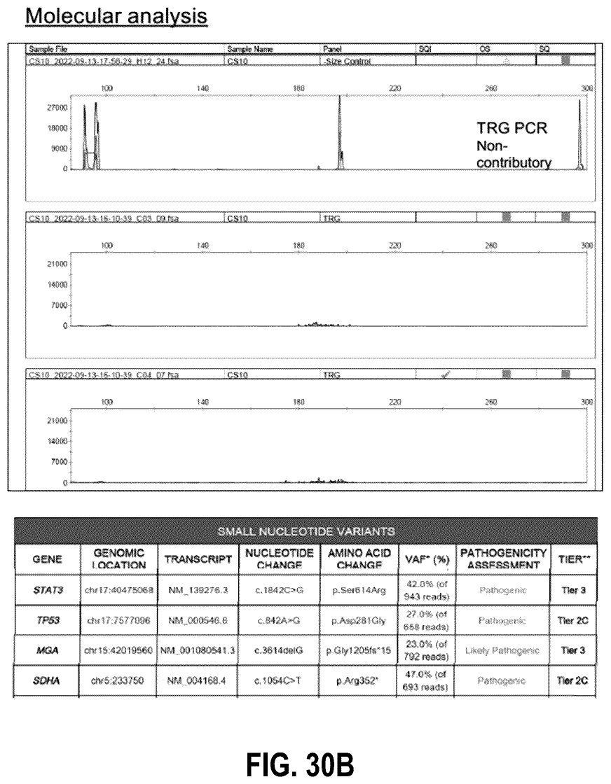

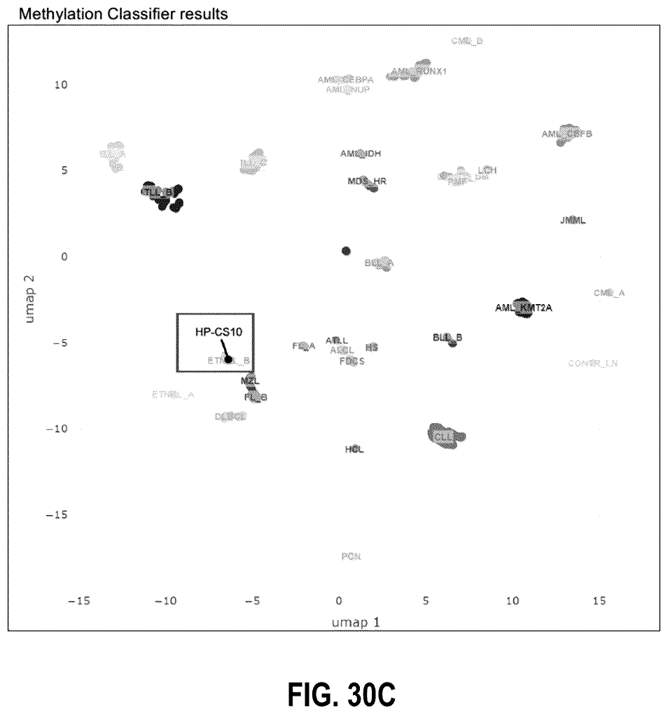

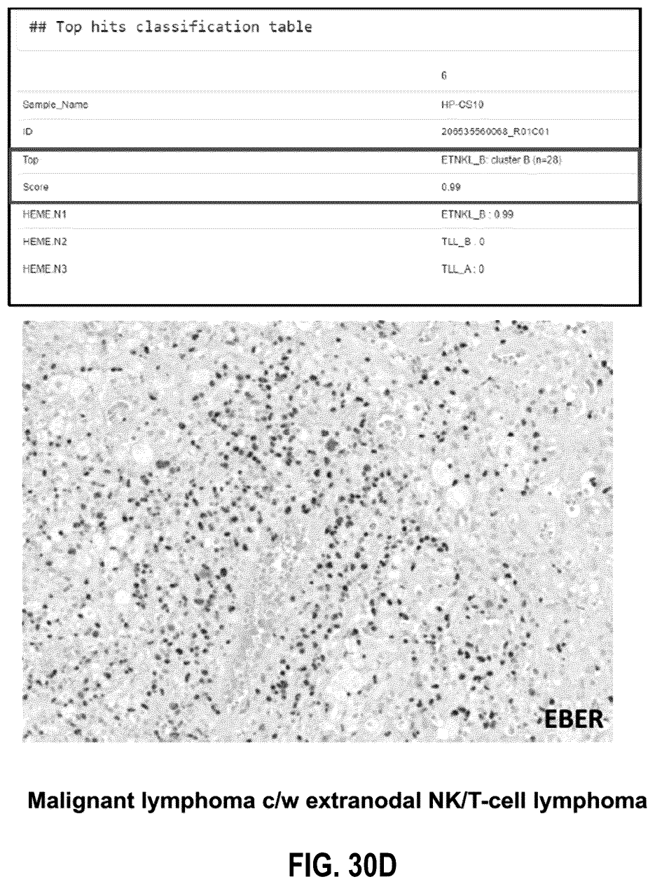

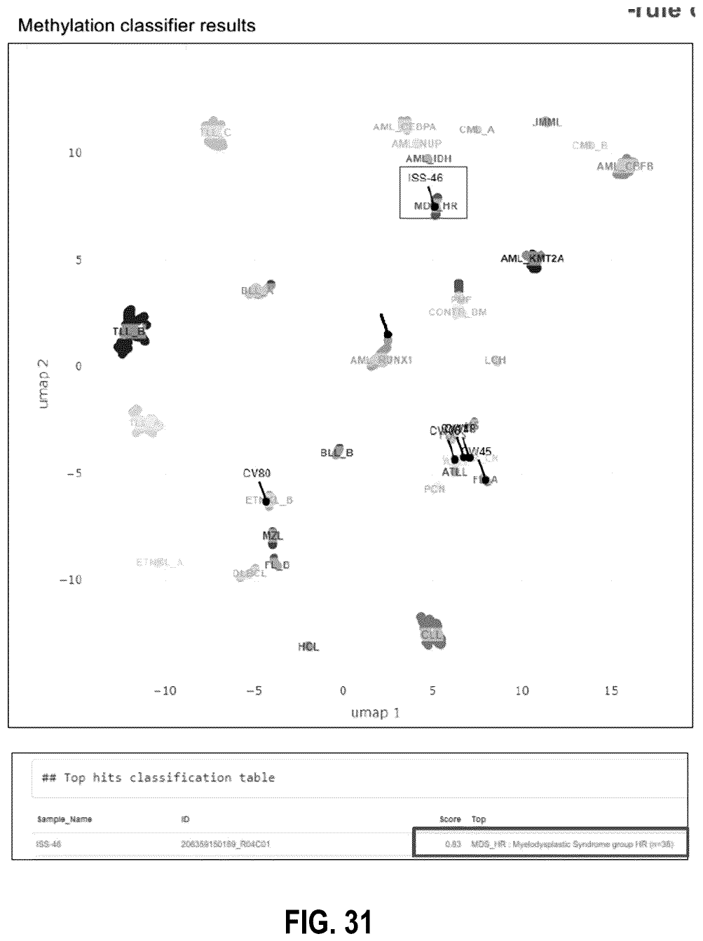

In an example, a brain biopsy from an 84 year old man with a brain lesion and no prior history of lymphadenopathy was classified using both standard pathology and the trained classifier to compare the classifications. FIGS. 30A-30B shows the results of classifying the brain biopsy. FIG. 30A shows morphology and immunohistochemistry (IHC) images that were acquired using standard techniques. FIG. 30A also includes a table of markers that the biopsy sample was positive or negative for. FIG. 30B shows results of the molecular analysis of the biopsy, including small nucleotide variants. The biopsy was identified as a peripheral T-cell lymphoma, consistent with ALK-negative ALCL. FIGS. 31C and 31D show the results of the methylation classifier, which classified the biopsy as a malignant lymphoma c/w extranodal NK/T-cell lymphoma.

In another example, a biopsy of a 73 year old female with a subdural mass was classified using both standard pathology and the trained classifier to compare the classifications. The lineage was undetermined because all markers were negative by IHC except weak focal CD45 such that HL tumor was ruled out. FIG. 31 shows the results of the methylation classifier, which classified the biopsy as a myeloid sarcoma/chloroma. This correlated with the PB/BM/clinical history. The case was transferred to hemepath while the patient was transferred to the ICU with hypercalcemia and blast crises, which corroborates with the methylation results).

DNA methylation profiling of hematolymphoid tumors may identify groups that correspond to known clinicopathologic entities, including tumor subgroups with clinical and biological relevance. The methylation classifier described herein is highly accurate for the HL classes that have been trained to date.

Having described several embodiments, it will be recognized by those skilled in the art that various modifications, alternative constructions, and equivalents may be used without departing from the spirit of the disclosure. Additionally, a number of well-known processes and elements have not been described in order to avoid unnecessarily obscuring the present disclosure. Accordingly, the above description should not be taken as limiting the scope of the disclosure.

Those skilled in the art will appreciate that the presently disclosed embodiments teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

### Exemplary Embodiments

The following is a list of non-limiting exemplary embodiments and may include combinations thereof.

Embodiment 1: A system for classifying a tumor, the system comprising: a processor in communication with a memory, the memory including instructions executable by the processor to: receive a methylation profile of the tumor; provide the methylation profile to a classifier trained to identify tumor classes using unsupervised clustering; generate a classification of the tumor based on the methylation profile and a reference set, wherein the reference set is generated from training the classifier; generate a confidence score based on the correlation of the methylation profile to the classification from the classifier; and update the classifier and reference set with the methylation profile and classification.

Embodiment 2: The system of embodiment 1, wherein the classifier comprises a plurality of sub-classifiers.

Embodiment 3: The system of embodiment 2, wherein the classifier comprises a plurality of family sub-classifiers for separate functional regions of the methylation profile.

Embodiment 4: The system of embodiment 3, wherein the classifier comprises at least 5 family sub-classifiers.

Embodiment 5: The system of embodiment 3, the memory further including instructions executable by the processor to: generate a family consistency score and a family mean calibrated score from the sub-classifiers.

Embodiment 6: The system of embodiment 5, the memory further including instructions executable by the processor to: generate a family classification based on the family consistency score and the family mean calibrated score.

Embodiment 7: The system of embodiment 6, wherein the classifier further comprises a class sub-classifier for each family.

Embodiment 8: The system of embodiment 7, wherein the classifier comprises at least 5 class sub-classifiers.

Embodiment 9: The system of embodiment 7, the memory further including instructions executable by the processor to: generate a class consistency score and a class mean calibrated score from the class sub-classifiers.

Embodiment 10: The system of embodiment 9, the memory further including instructions executable by the processor to: generate a class and/or sub-class classification based on the class consistency score and the class mean calibrated score.

Embodiment 11: The system of embodiment 10, wherein the confidence score comprises a mean calibrated score of the family consistency score, the family mean calibrated score, the class consistency score, and/or the class mean calibrated score.

Embodiment 12: The system of embodiment 1, the memory further including instructions executable by the processor to: identify a tumor family, class, and sub-class based on the classification of the tumor.

Embodiment 13: The system of embodiment 12, wherein the tumor sub-class is identified based on clusters of characteristics identified by the classifier.

Embodiment 14: The system of embodiment 13, wherein the tumor is a renal tumor, hematolymphoid tumor, or CNS tumor.

Embodiment 15: The system of embodiment 1, wherein when the confidence score is above a threshold, there is high confidence in the classification.

Embodiment 16: The system of embodiment 15, wherein the threshold is at least 0.9.

Embodiment 17: The system of embodiment 1, wherein when the confidence score is below 0.5 or the classifier cannot generate a classification, the memory further including instructions executable by the processor to: generate an alert for a new class or sub-class.

Embodiment 18: The system of embodiment 17, the memory further including instructions executable by the processor to: evaluate the methylation profile, a sample of the tumor, orthogonal DNA and/or RNA data, and/or patient demographics to generate the new class or sub-class.

Embodiment 19: The system of embodiment 18, the memory further including instructions executable by the processor to: update the reference set with the new class or sub-class.

Embodiment 20: The system of embodiment 19, the memory further including instructions executable by the processor to: re-train the classifier with the updated reference set.

Embodiment 21: The system of embodiment 1, wherein the unsupervised clustering uses uniform manifold approximation and projection (UMAP) dimensionality reduction and/or additional dimensionality reduction methodologies.

Embodiment 22: The system of embodiment 1, the memory further including instructions executable by the processor to: train the classifier prior to providing the methylation profile.

Embodiment 23: The system of embodiment 1, the memory further including instructions executable by the processor to: diagnose the tumor using the generated classification.

Embodiment 24: The system of embodiment 23, the memory further including instructions executable by the processor to: form a treatment plan specific to the diagnosis of the tumor.

Embodiment 25: The system of embodiment 24, the memory further including instructions executable by the processor to: compare the classification to a histological and/or molecular evaluation of the tumor.

Embodiment 26: The system of embodiment 25, the memory further including instructions executable by the processor to: adjust the classification based on the comparison.

Embodiment 27: The system of embodiment 22, the memory further including instructions executable by the processor to: adjust the classification based on demographic data or other DNA and/or RNA data of the patient.

Embodiment 28: A system for training a classifier for classifying a tumor, the system comprising: a processor in communication with a memory, the memory including instructions executable by the processor to: receive a reference methylation dataset comprising a plurality of methylation profiles for a plurality of samples; apply unsupervised clustering to the methylation dataset using a cluster model; filter the samples based on clustering of the methylation profiles to identify families of the tumor; apply unsupervised clustering using the most variable probes from different functional regions on each methylation profile in the methylation dataset for a plurality of sub-classifiers to identify classes and sub-classes of the tumor based on clusters of methylation profiles; and generate a reference set based on the identified families, classes, and sub-classes.

Embodiment 29: The system of embodiment 28, wherein the unsupervised clustering uses Uniform Manifold Approximation and Projection (UMAP) dimensionality reduction and/or additional dimensionality reduction methodologies.

Embodiment 30: The system of embodiment 28, wherein the different functional regions are selected from the group consisting of Gene body, Island, Opensea, Other genomic region, Open chromatin region, Shore, Shelf, TSS, 5′UTR, and whole array probes.

Embodiment 31: The system of embodiment 28, the memory further including instructions executable by the processor to: implement a support vector machine (SVM) algorithm for each classifier.

Embodiment 32: The system of embodiment 28, the memory further including instructions executable by the processor to: apply a 5 by 5 nested cross-validation (CV).

Embodiment 33: The system of embodiment 28, the memory further including instructions executable by the processor to: calibrate each classifier.

Embodiment 34: The system of embodiment 28, the memory further including instructions executable by the processor to: update the classification database with new methylation profiles of new samples.

Embodiment 35: The system of embodiment 34, the memory further including instructions executable by the processor to: re-train the classifier with the updated reference set.

Embodiment 36: A method of recommending a treatment plan for the patient, the method comprising: diagnosing the tumor using the classification generated from any one of the preceding claims.

Embodiment 37: The method of embodiment 36, further comprising forming a treatment plan specific to the diagnosis of the tumor.

Embodiment 38: The method of embodiment 37, further comprising comparing the classification to a histological and/or molecular evaluation of the tumor.

Embodiment 39: The method of embodiment 38, further comprising adjusting the classification based on the comparison.

Embodiment 40: The method of embodiment 36, further comprising adjusting the classification based on demographic data or other DNA and/or RNA data of the patient.

Embodiment 41: A method of training a classifier for classifying a tumor, the method comprising: providing a reference methylation dataset comprising a plurality of methylation profiles for a plurality of samples; applying unsupervised clustering to the methylation dataset; filtering the samples based on clustering of the methylation profiles to identify families of the tumor; applying unsupervised clustering using the most variable probes from different functional regions on each methylation profile in the methylation dataset for a plurality of sub-classifiers to identify classes and sub-classes of the tumor based on clusters of methylation profiles; and generating a reference set based on the identified families, classes, and sub-classes.

Embodiment 42: The method of embodiment 41, wherein the unsupervised clustering uses Uniform Manifold Approximation and Projection (UMAP) dimensionality reduction and/or additional dimensionality reduction methodologies.

Embodiment 43: The method of embodiment 41, wherein the different functional regions are selected from the group consisting of Gene body, Island, Opensea, Other genomic region, Open chromatin region, Shore, Shelf, TSS, 5′UTR, and whole array probes.

Embodiment 44: The method of embodiment 41, further comprising implementing a support vector machine (SVM) algorithm for each classifier.

Embodiment 45: The method of embodiment 41, further comprising applying a 5 by 5 nested cross-validation (CV).

Embodiment 46: The method of embodiment 41, further comprising calibrating each classifier.

Embodiment 47: The method of embodiment 41, further comprising updating the classification database with new methylation profiles of new samples.

Embodiment 48: A non-transitory computer readable medium storing instructions that when executed by at least one processor, cause the at least one processor to perform operations for device management, the operations comprising: receiving a methylation profile to a classifier trained to identify tumor classes using unsupervised clustering; generating a classification of the tumor based on the methylation profile and a reference set, wherein the reference set is generated from training the classifier; and generating a confidence score based on the correlation of the methylation profile to the classification from the classifier; and updating the classifier and reference set with the methylation profile and classification.

Embodiment 49: The non-transitory computer readable medium of embodiment 48, wherein the classifier comprises a plurality of sub-classifiers.

Embodiment 50: The non-transitory computer readable medium of embodiment 49, wherein the classifier comprises a plurality of family sub-classifiers for separate functional regions of the methylation profile.

Embodiment 51: The non-transitory computer readable medium of embodiment 50, the operations further comprising generating a family consistency score and a family mean calibrated score from the sub-classifiers.

Embodiment 52: The non-transitory computer readable medium of embodiment 51, the operations further comprising generating a family classification based on the family consistency score and the family mean calibrated score.

Embodiment 53: The non-transitory computer readable medium of embodiment 52, wherein the classifier further comprises a class sub-classifier for each family.

Embodiment 54: The non-transitory computer readable medium of embodiment 53, the operations further comprising generating a class consistency score and a class mean calibrated score from the class sub-classifiers.

Embodiment 55: The non-transitory computer readable medium of embodiment 54, the operations further comprising generating a class and/or sub-class classification based on the class consistency score and the class mean calibrated score.

Embodiment 56: The non-transitory computer readable medium of embodiment 55, wherein the confidence score comprises a mean calibrated score of the family consistency score, the family mean calibrated score, the class consistency score, and/or the class mean calibrated score.

Embodiment 57: The non-transitory computer readable medium of embodiment 48, the operations further comprising identifying a tumor family, class, and sub-class based on the classification of the tumor.

Embodiment 58: The non-transitory computer readable medium of embodiment 57, wherein the tumor sub-class is identified based on clusters of characteristics identified by the classifier.

Embodiment 59: The non-transitory computer readable medium of embodiment 48, wherein when the confidence score is above a threshold, there is high confidence in the classification.

Embodiment 60: The non-transitory computer readable medium of embodiment 59, wherein the threshold is at least 0.9.

Embodiment 61: The non-transitory computer readable medium of embodiment 48, wherein when the confidence score is below 0.5 or the classifier cannot generate a classification, the operations further comprise generating an alert for a new class or sub-class.

Embodiment 62: The non-transitory computer readable medium of embodiment 61 the operations further comprising generating the new class or sub-class.

Embodiment 63: The non-transitory computer readable medium of embodiment 62, the operations further comprising updating the reference set with the new class or sub-class.

Embodiment 64: The non-transitory computer readable medium of embodiment 48, wherein the unsupervised clustering uses uniform manifold approximation and projection (UMAP) dimensionality reduction and/or additional dimensionality reduction methodologies.

Embodiment 65: The non-transitory computer readable medium of embodiment 48, the operations further comprising training the classifier prior to receiving the methylation profile.

What is claimed is:

1. A system for classifying a tumor, the system comprising:
a processor in communication with a memory, the memory including instructions executable by the processor to:
receive a methylation profile of the tumor;
provide the methylation profile to a classifier trained to identify tumor classes using unsupervised clustering;
generate a classification of the tumor based on the methylation profile and a reference set, wherein the reference set is generated from training the classifier;
generate a confidence score based on the correlation of the methylation profile to the classification from the classifier; and
update the classifier and reference set with the methylation profile and classification.

2. The system of claim 1, wherein the classifier comprises a plurality of sub-classifiers.

3. The system of claim 2, wherein the classifier comprises a plurality of family sub-classifiers for separate functional regions of the methylation profile.

4. The system of claim 3, the memory further including instructions executable by the processor to: generate a family consistency score and a family mean calibrated score from the sub-classifiers.

5. The system of claim 4, the memory further including instructions executable by the processor to: generate a family classification based on the family consistency score and the family mean calibrated score.

6. The system of claim 5, wherein the classifier further comprises a class sub-classifier for each family.

7. The system of claim 6, the memory further including instructions executable by the processor to: generate a class consistency score and a class mean calibrated score from the class sub-classifiers.

8. The system of claim 7, the memory further including instructions executable by the processor to: generate a class and/or sub-class classification based on the class consistency score and the class mean calibrated score.

9. The system of claim 8, wherein the confidence score comprises a mean calibrated score of the family consistency score, the family mean calibrated score, the class consistency score, and/or the class mean calibrated score.

10. The system of claim 1, the memory further including instructions executable by the processor to: identify a tumor family, class, and sub-class based on clusters of characteristics identified by the classifier.

11. The system of claim 10, wherein the tumor is a renal tumor, hematolymphoid tumor, or CNS tumor.

12. The system of claim 1, wherein there is high confidence in the classification when the confidence score is above a threshold of at least 0.9.

13. The system of claim 1, wherein when the confidence score is below 0.5 or the classifier cannot generate a classification, the memory further including instructions executable by the processor to: generate an alert for a new class or sub-class.

14. The system of claim 13, the memory further including instructions executable by the processor to: evaluate the methylation profile, a sample of the tumor, orthogonal DNA and/or RNA data, and/or patient demographics to generate the new class or sub-class.

15. The system of claim 14, the memory further including instructions executable by the processor to: update the reference set with the new class or sub-class and re-train the classifier with the updated reference set.

16. The system of claim 1, wherein the unsupervised clustering uses uniform manifold approximation and projection (UMAP) dimensionality reduction and/or additional dimensionality reduction methodologies.

17. The system of claim 1, the memory further including instructions executable by the processor to: train the classifier prior to providing the methylation profile.

18. The system of claim 1, the memory further including instructions executable by the processor to: diagnose the tumor using the generated classification.

19. The system of claim 18, the memory further including instructions executable by the processor to: form a treatment plan specific to the diagnosis of the tumor.

20. The system of claim 19, the memory further including instructions executable by the processor to: compare the classification to a histological and/or molecular evaluation of the tumor and adjust the classification based on the comparison.
